(19) United States
(12) Patent Application Publication
(10) Pub. No.: US 2023/0172521 A1
(43) Pub. Date: Jun. 8, 2023

(54) DETERMINATION OF CARDIAC CONDUCTION SYSTEM THERAPY BENEFIT

(71) Applicant: Medtronic, Inc., Minneapolis, MN (US)

(72) Inventor: Subham Ghosh, Blaine, MN (US)

(21) Appl. No.: 17/969,961

(22) Filed: Oct. 20, 2022

Related U.S. Application Data
(60) Provisional application No. 63/286,908, filed on Dec. 7, 2021.

(51) Int. Cl. A61B 5/367; A61B 5/339; A61B 5/00; A61B 5/282

(52) U.S. Cl. CPC A61B 5/367; A61B 5/339; A61B 5/7475; A61B 5/282

(57) ABSTRACT

Determination of cardiac conduction system pacing therapy benefit may be performed by the systems, methods, devices, and interfaces described herein. For example, various metrics of activation time dispersion may be generated based on electrical activity monitored by a plurality of external electrodes such as, e.g., a left-sided metric of dispersion and a global metric of dispersion. Such various metrics of activation time dispersion may be used to determined whether cardiac conduction system pacing would be beneficial.

### RELATED APPLICATIONS

This application claims the benefit of U.S. Provisional Application Ser. No. 63/286,908, filed Dec. 7, 2021, the entire contents of each of which are incorporated herein by reference.

### TECHNICAL FIELD

The disclosure herein relates to systems and methods for use in determining cardiac conduction system therapy benefit using a plurality of external electrodes.

### BACKGROUND

Implantable medical devices (IMDs), such as implantable pacemakers, cardioverters, defibrillators, or pacemaker-cardioverter-defibrillators, provide therapeutic electrical stimulation to the heart. IMDs may provide pacing to address bradycardia, or pacing or shocks in order to terminate tachyarrhythmia, such as tachycardia or fibrillation. In some cases, the medical device may sense intrinsic depolarizations of the heart, detect arrhythmia based on the intrinsic depolarizations (or absence thereof), and control delivery of electrical stimulation to the heart based on the intrinsic depolarizations.

IMDs may also provide cardiac resynchronization therapy (CRT), which is a form of pacing. CRT involves the delivery of pacing to the left ventricle, or both the left and right ventricles. The timing and location of the delivery of pacing pulses to the ventricle(s) may be selected to improve the coordination and efficiency of ventricular contraction.

IMDs may be described as delivering one or both of conventional pacing therapy and cardiac conduction system pacing therapy. Conventional, or traditional, pacing therapy may be described as delivering pacing pulses into myocardial tissue that is not part of the cardiac conduction system of the patient's heart such that, e.g., the pacing pulses trigger electrical activation that propagates primarily from one myocardial cell to another myocardial cell (also referred to as “cell-to-cell”) as opposed to propagating within the cardiac conduction system prior to the myocardial tissue. For instance, conventional pacing therapy may deliver pacing pulses directly into the muscular heart tissue that is to be depolarized to provide the contraction of the heart. For example, conventional left ventricular pacing therapy may utilize a left ventricular (LV) coronary sinus lead that is implanted so as to extend through one or more veins, the vena cava, the right atrium, and into the coronary sinus to a region adjacent to the free wall of the left ventricle of the heart so as to deliver pacing pulses to the myocardial tissue of the free wall of the left ventricle.

Cardiac conduction system pacing therapy may be described as delivering pacing pulses into the cardiac conduction system through pacing electrodes positioned proximate or in direct contact with one or more portions or regions of the cardiac conduction system. The cardiac conduction system may include one or more parts of the cardiac conduction system such as the left bundle branch, bundle of His, atrioventricular node, right bundle branch, etc. Thus, cardiac conduction system pacing therapy may utilize or include one or more electrodes positioned proximate or in direct contact with the left bundle branch, bundle of His, atrioventricular node, right bundle branch, etc. to deliver pacing pulses into and within the cardiac conduction system. For example, a ventricle-from-atrium (VfA) lead may deliver pacing pulses directly to the left bundle branch of the cardiac conduction system such that the pulses propagate along the left bundle branch and Purkinje fibers to initiate depolarization of cardiac tissues proximate thereto (e.g., the myocardial tissue of the left ventricle).

Systems for implanting medical devices may include workstations or other equipment in addition to the implantable medical device itself. In some cases, this equipment assists the physician or other technician with placing the intracardiac leads at particular locations on the heart. In some cases, the equipment provides information to the physician about the electrical activity of the heart and the location of the intracardiac lead. The equipment may perform similar functions as the medical device, including delivering electrical stimulation to the heart and sensing the depolarizations of the heart. In some cases, the equipment may include equipment for obtaining electrocardiograms (ECGs) via electrodes on the surface, or skin, of the patient. More specifically, the patient may have a plurality of electrodes on an ECG belt or vest that surrounds the torso of the patient. After the belt or vest has been secured to the torso, a physician can perform a series of tests to evaluate a patient's cardiac response. The evaluation process can include, among other things, detection of a baseline rhythm in which no electrical stimuli is delivered to cardiac tissue. Further, electrodes placed on the body surface of the patient may be used for various therapeutic purposes (e.g., cardiac resynchronization therapy) including optimizing lead location, pacing parameters, etc. based on one or more metrics derived from the signals captured by the ECG electrodes.

### SUMMARY

The exemplary systems and methods described herein may be configured to assist users (e.g., physicians, clinicians, doctors, etc.) to determine whether a patient may benefit from cardiac conduction system therapy or conventional cardiac pacing therapy (e.g., left ventricular myocardial pacing therapy) prior to implantation and configuration of cardiac therapy apparatus to perform one or both of cardiac conduction system pacing therapy and conventional cardiac pacing therapy. Thus, the illustrative systems and methods may be performed during intrinsic activation of the patient's heart (e.g., without any cardiac therapy being delivered to the patient and allowing the patient's heart to beat naturally). Further, the systems and methods may be described as being noninvasive. For example, the systems and methods may not use implantable devices such as leads, probes, sensors, catheters, etc. to evaluate whether the patient may benefit from the cardiac conduction system therapy or to determine the location or position of a cardiac conduction system block. Instead, the systems and methods may use electrical measurements taken noninvasively using, e.g., a plurality of external electrodes attached to the skin of a patient about the patient's torso. It may be described that the illustrative systems and methods may provide a screening system to determine which patients may likely benefit from cardiac conduction system pacing ahead of any invasive procedure to implantation a cardiac conduction system pacing apparatus based on intrinsic ECG maps and metrics derived therefrom.

One illustrative system may include, among other things, a computing apparatus comprising processing circuitry. The computing apparatus may be configured to obtain external electrical activity measured from tissue of a patient during intrinsic activation of the patient's heart and generate electrical heterogeneity information (EHI) based on the obtained electrical activity. The EHI may include one or more metrics of dispersion of cardiac electrical activation times. The computing apparatus may be further configured to determine that cardiac conduction system pacing therapy would benefit the patient based on at least the one or more metrics of dispersion of cardiac electrical activation times.

One illustrative method may include obtaining external electrical activity measured from tissue of a patient during intrinsic activation of the patient's heart and generating electrical heterogeneity information (EHI) based on the obtained electrical activity. The EHI may include one or more metrics of dispersion of cardiac electrical activation times. The method may further comprise determining that cardiac conduction method pacing therapy would benefit the patient based on at least the one or more metrics of dispersion of cardiac electrical activation times.

One illustrative system may include, among other things, a computing apparatus comprising processing circuitry. The computing apparatus may be configured to generate one or more metrics of dispersion of cardiac electrical activation times based on a plurality of surrogate cardiac electrical activation times representative of depolarization of a plurality of regions of a patient's heart during intrinsic activation, and determine that cardiac conduction system pacing therapy would benefit the patient based on at least the one or more metrics of dispersion of cardiac electrical activation times.

The above summary is not intended to describe each embodiment or every implementation of the present disclosure. A more complete understanding will become apparent and appreciated by referring to the following detailed description and claims taken in conjunction with the accompanying drawings.

### BRIEF DESCRIPTION OF THE DRAWINGS

FIG. 1 is a diagram of an illustrative system including electrode apparatus, display apparatus, and computing apparatus.

FIGS. 2-3 are diagrams of illustrative external electrode apparatus for measuring torso-surface potentials.

FIG. 4A depicts a patient's cardiac conduction network including a cardiac conduction system block positioned between the atrioventricular node and the bundle of His.

FIG. 4B depicts a patient's cardiac conduction network including a cardiac conduction system block positioned in the left branch.

FIG. 5A is a block diagram of an illustrative method for determining whether a patient may benefit from cardiac conduction system pacing therapy.

FIG. 5B is a detailed block diagram of an illustrative method of the method depicted in FIG. 5A.

FIG. 6 is a scatterplot of the standard deviation of activation times and left-sided standard deviation of activation times for a plurality of patients.

FIG. 7 is a conceptual diagram of an illustrative cardiac therapy system including an intracardiac medical device implanted in a patient's heart and a separate medical device positioned outside of the patient's heart.

FIG. 8 is an enlarged conceptual diagram of the intracardiac medical device of FIG. 7 and anatomical structures of the patient's heart.

FIG. 9 is a conceptual diagram of a map of a patient's heart in a standard 17 segment view of the left ventricle showing various electrode implantation locations for use with the illustrative systems and devices described herein.

FIG. 10 is a block diagram of illustrative circuitry that may be enclosed within the housing of the medical devices of FIGS. 7-8, for example, to provide the functionality and therapy described herein.

### DETAILED DESCRIPTION OF ILLUSTRATIVE EMBODIMENTS

In the following detailed description of illustrative embodiments, reference is made to the accompanying figures of the drawing which form a part hereof, and in which are shown, by way of illustration, specific embodiments which may be practiced. It is to be understood that other embodiments may be utilized and structural changes may be made without departing from (e.g., still falling within) the scope of the disclosure presented hereby.

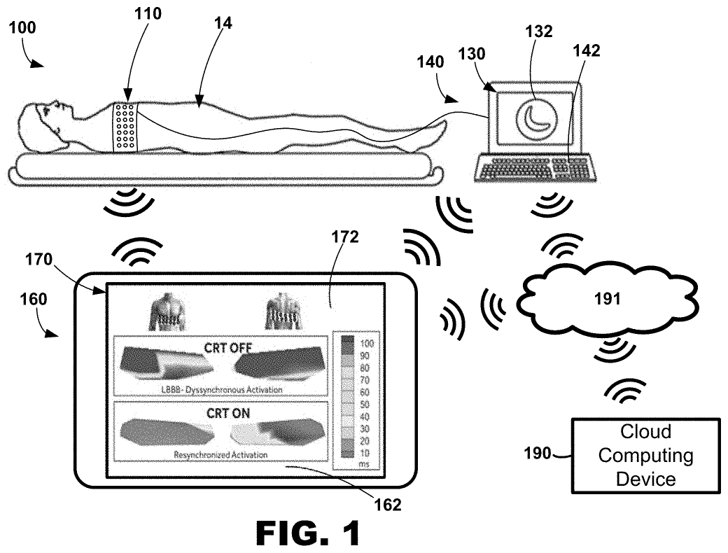

Illustrative systems and methods shall be described with reference to FIGS. 1-10. It will be apparent to one skilled in the art that elements or processes from one embodiment may be used in combination with elements or processes of the other embodiments, and that the possible embodiments of such systems and methods using combinations of features set forth herein is not limited to the specific embodiments shown in the Figures and/or described herein. Further, it will be recognized that the embodiments described herein may include many elements that are not necessarily shown to scale. Still further, it will be recognized that timing of the processes and the size and shape of various elements herein may be modified but still fall within the scope of the present disclosure, although certain timings, one or more shapes and/or sizes, or types of elements, may be advantageous over others.

A plurality of electrocardiogram (ECG) signals (e.g., torso-surface potentials) may be measured, or monitored, using a plurality of external electrodes positioned about the surface, or skin, of a patient. The ECG signals may be used to evaluate a patient's cardiac health, to determine whether the patient may benefit from cardiac conduction pacing therapy and/or another cardiac therapy, and to determine the location or relative position of a cardiac conduction system block. As described herein, the ECG signals may be gathered or obtained noninvasively since, e.g., implantable electrodes may not be used to measure the ECG signals. Further, the ECG signals may be used to determine cardiac electrical activation times, which may be used to generate various metrics (e.g., electrical heterogeneity information) that may be used (and initiated by a user such as a physician) to determine whether a patient may benefit from cardiac pacing therapy such as, e.g., cardiac conduction system pacing therapy and/or conventional pacing therapy.

Various illustrative systems, methods, devices, and graphical user interfaces provided thereby may be configured to use electrode apparatus including external electrodes, display apparatus, and computing apparatus to noninvasively assist a user (e.g., a physician) evaluation of cardiac health, in the determination of a location of cardiac conduction system block, and in the determination of whether a patient may benefit from cardiac conduction system pacing therapy and/or another type of cardiac therapy. An illustrative system 100 including electrode apparatus 110 and computing apparatus is depicted in FIG. 1. The computing apparatus may include, among other things, a local computing device 140, a remote computing device 160, and a cloud computing device 190. It is to be understood that the computing apparatus may include any one of the local computing device 140, the remote computing device 160, and the cloud computing device 190 or any combination of the local computing device 140, the remote computing device 160, and the cloud computing device 190 operating in conjunction with each other.

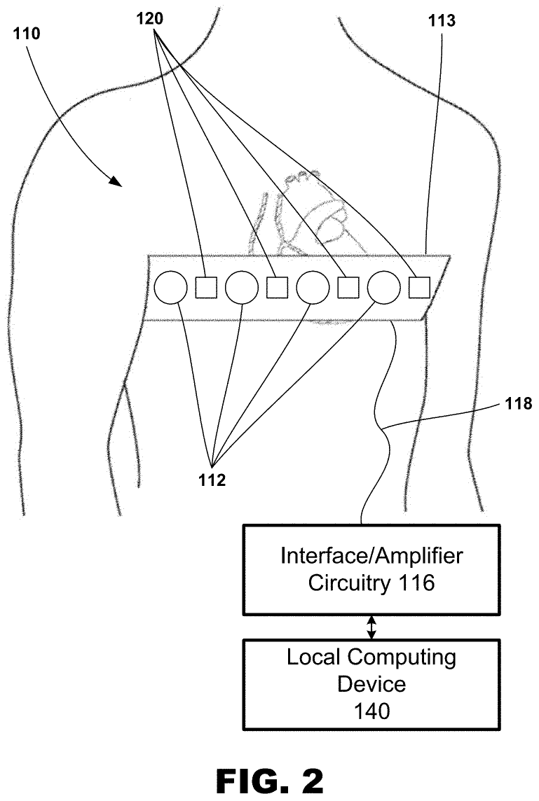

The electrode apparatus 110 as shown includes a plurality of electrodes incorporated, or included, within a band wrapped around the chest, or torso, of a patient 14. The electrode apparatus 110 is operatively coupled to the local computing device 140 (e.g., through one or wired electrical connections, wirelessly, etc.) to provide electrical signals from each of the electrodes to the local computing device 140 for analysis, evaluation, etc. Illustrative electrode apparatus may be described in U.S. Pat. No. 9,320,446 entitled “Bioelectric Sensor Device and Methods” filed Mar. 27, 2014, and issued on Mar. 26, 2016, which is incorporated herein by reference in its entirety. Further, illustrative electrode apparatus 110 will be described in more detail in reference to FIGS. 2-3.

Although not described herein, the illustrative system 100 may further include imaging apparatus. The imaging apparatus may be any type of imaging apparatus configured to image, or provide images of, at least a portion of the patient in a noninvasive manner. For example, the imaging apparatus may not use any components or parts that may be located within the patient to provide images of the patient except noninvasive tools such as contrast solution. It is to be understood that the illustrative systems, methods, devices, and interfaces described herein may further use imaging apparatus to provide noninvasive assistance to a user (e.g., a physician) to locate, or place, one or more pacing electrodes proximate the patient's heart in conjunction with the configuration of cardiac therapy.

For example, the illustrative systems, methods, devices, and interfaces may provide image guided navigation that may be used to navigate leads including electrodes, leadless electrodes, wireless electrodes, catheters, etc., within the patient's body while also providing noninvasive cardiac therapy configuration including determination of an effective, or optimal, pre-excitation intervals such as A-V and V-V intervals, etc. Illustrative systems, methods, devices, and interfaces that use imaging apparatus and/or electrode apparatus may be described in U.S. Pat. App. Pub. No. 2014/0371832 to Ghosh published on Dec. 18, 2014, U.S. Pat. App. Pub. No. 2014/0371833 to Ghosh et al. published on Dec. 18, 2014, U.S. Pat. App. Pub. No. 2014/0323892 to Ghosh et al. published on Oct. 30, 2014, U.S. Pat. App. Pub. No. 2014/0323882 to Ghosh et al. published on Oct. 20, 2014, each of which is incorporated herein by reference in its entirety.

Illustrative imaging apparatus may be configured to capture x-ray images and/or any other alternative imaging modality. For example, the imaging apparatus may be configured to capture images, or image data, using isocentric fluoroscopy, bi-plane fluoroscopy, ultrasound, computed tomography (CT), multi-slice computed tomography (MSCT), magnetic resonance imaging (MRI), high frequency ultrasound (HIFU), optical coherence tomography (OCT), intra-vascular ultrasound (IVUS), two dimensional (2D) ultrasound, three dimensional (3D) ultrasound, four dimensional (4D) ultrasound, intraoperative CT, intraoperative MRI, etc. Further, it is to be understood that the imaging apparatus may be configured to capture a plurality of consecutive images (e.g., continuously) to provide video frame data. In other words, a plurality of images taken over time using the imaging apparatus may provide video frame, or motion picture, data. An exemplary system that employs ultrasound can be found in U.S. Pat. App. Pub. No. 2017/0303840 to Stadler et al. published on Oct. 26, 2017, which is incorporated by reference in its entirety. Additionally, the images may also be obtained and displayed in two, three, or four dimensions. In more advanced forms, four-dimensional surface rendering of the heart or other regions of the body may also be achieved by incorporating heart data or other soft tissue data from a map or from pre-operative image data captured by MRI, CT, or echocardiography modalities. Image datasets from hybrid modalities, such as positron emission tomography (PET) combined with CT, or single photon emission computer tomography (SPECT) combined with CT, could also provide functional image data superimposed onto anatomical data, e.g., to be used to navigate implantable apparatus to target locations within the heart or other areas of interest.

Systems and/or imaging apparatus that may be used in conjunction with the illustrative systems, methods, devices, and interfaces described herein are described in U.S. Pat. App. Pub. No. 2005/0008210 to Evron et al. published on Jan. 13, 2005, U.S. Pat. App. Pub. No. 2006/0074285 to Zarkh et al. published on Apr. 6, 2006, U.S. Pat. No. 8,731,642 to Zarkh et al. issued on May 20, 2014, U.S. Pat. No. 8,861,830 to Brada et al. issued on Oct. 14, 2014, U.S. Pat. No. 6,980,675 to Evron et al. issued on Dec. 27, 2005, U.S. Pat. No. 7,286,866 to Okerlund et al. issued on Oct. 23, 2007, U.S. Pat. No. 7,308,297 to Reddy et al. issued on Dec. 11, 2011, U.S. Pat. No. 7,308,299 to Burrell et al. issued on Dec. 11, 2011, U.S. Pat. No. 7,321,677 to Evron et al. issued on Jan. 22, 2008, U.S. Pat. No. 7,346,381 to Okerlund et al. issued on Mar. 18, 2008, U.S. Pat. No. 7,454,248 to Burrell et al. issued on Nov. 18, 2008, U.S. Pat. No. 7,499,743 to Vass et al. issued on Mar. 3, 2009, U.S. Pat. No. 7,565,190 to Okerlund et al. issued on Jul. 21, 2009, U.S. Pat. No. 7,587,074 to Zarkh et al. issued on Sep. 8, 2009, U.S. Pat. No. 7,599,730 to Hunter et al. issued on Oct. 6, 2009, U.S. Pat. No. 7,613,500 to Vass et al. issued on Nov. 3, 2009, U.S. Pat. No. 7,742,629 to Zarkh et al. issued on Jun. 22, 2010, U.S. Pat. No. 7,747,047 to Okerlund et al. issued on Jun. 29, 2010, U.S. Pat. No. 7,778,685 to Evron et al. issued on Aug. 17, 2010, U.S. Pat. No. 7,778,686 to Vass et al. issued on Aug. 17, 2010, U.S. Pat. No. 7,813,785 to Okerlund et al. issued on Oct. 12, 2010, U.S. Pat. No. 7,996,063 to Vass et al. issued on Aug. 9, 2011, U.S. Pat. No. 8,060,185 to Hunter et al. issued on Nov. 15, 2011, and U.S. Pat. No. 8,401,616 to Verard et al. issued on Mar. 19, 2013, each of which is incorporated herein by reference in its entirety.

The local computing device 140, the remote computing device 160, and the cloud computing device 190 may be configured to monitor (e.g., using the electrode apparatus), generate, and analyze data such as, e.g., electrical signals (e.g., electrocardiogram data), electrical activation times, electrical heterogeneity information, etc. For example, one cardiac cycle, or one heartbeat, of a plurality of cardiac cycles, or heartbeats, represented by the electrical signals collected or monitored by the electrode apparatus 110 may be analyzed and evaluated for data or information that may be pertinent to determining whether a patient would benefit from one or more different types of cardiac therapy such as cardiac conduction system pacing therapy. Additionally, such surrogate cardiac electrical activation times and electrical heterogeneity information may also be pertinent to the therapeutic nature of one or more parameters related to cardiac therapy such as, e.g., pacing parameters, lead location, etc., and thus, may be useful for the adjustment thereof. More specifically, for example, the QRS complex of a single cardiac cycle may be evaluated for one or more metrics such as, e.g., QRS onset, QRS offset, QRS peak, electrical activation times referenced to earliest activation time, electrical heterogeneity information (EHI) such as left ventricular or thoracic standard deviation of electrical activation times (LVED), standard deviation of activation times (SDAT), and average left ventricular or thoracic surrogate electrical activation times (LVAT), QRS duration (e.g., interval between QRS onset to QRS offset), difference between average left surrogate and average right surrogate activation times, relative or absolute QRS morphology, difference between a higher percentile and a lower percentile of activation times (higher percentile may be 90%, 80%, 75%, 70%, etc. and lower percentile may be 10%, 15%, 20%, 25% and 30%, etc.), other statistical measures of central tendency (e.g., median or mode), dispersion (e.g., mean deviation, standard deviation, variance, interquartile deviations, range, etc.), etc. Further, each of the one or more metrics may be location specific. For example, some metrics may be computed from signals recorded, or monitored, from electrodes positioned about a selected area of the patient such as, e.g., the left side of the patient, the right side of the patient, etc. Additionally, the local computing device 140 and the remote computing device 160 may each include display apparatus 130, 170, respectively, that may be configured to display such data.

In at least one embodiment, each of the local computing device 140, the remote computing device 160, and the cloud computing device 190 may be a server, a personal computer, a tablet computer, a mobile device, and a cellular telephone. The local computing device 140 may be configured to receive input from input apparatus 142 (e.g., a keyboard) and transmit output to the display apparatus 130, and the remote computing device 160 may be configured to receive input from input apparatus 162 (e.g., a touchscreen) and transmit output to the display apparatus 170. The local computing device 140, the remote computing device 160, and the cloud computing device 190 may include data storage that may allow for access to processing programs or routines and/or one or more other types of data, e.g., for analyzing a plurality of electrical signals captured by the electrode apparatus 110, for determining QRS onsets, QRS offsets, medians, modes, averages, peaks or maximum values, valleys or minimum values in such electrical signals, for determining electrical activation times, for driving graphical user interfaces configured to noninvasively assist a user in determining whether a patient may benefit from cardiac conduction system pacing therapy and/or another cardiac therapy, for driving graphical user interfaces configured to noninvasively assist a user in determining the location or relative position of a cardiac conduction system block, for configuring one or more pacing parameters, or settings, such as, e.g., pacing rate, ventricular pacing rate, A-V interval, V-V interval, pacing pulse width, pacing vector, multipoint pacing vector (e.g., left ventricular vector quad lead), pacing voltage, pacing configuration (e.g., biventricular pacing, right ventricle only pacing, left ventricle only pacing, etc.), etc.

The local computing device 140 may be operatively coupled to the input apparatus 142 and the display apparatus 130 to, e.g., transmit data to and from each of the input apparatus 142 and the display apparatus 130, and the remote computing device 160 may be operatively coupled to the input apparatus 162 and the display apparatus 170 to, e.g., transmit data to and from each of the input apparatus 162 and the display apparatus 170. For example, the local computing device 140 and the remote computing device 160 may be electrically coupled to the input apparatus 142, 162 and the display apparatus 130, 170 using, e.g., analog electrical connections, digital electrical connections, wireless connections, bus-based connections, network-based connections, internet-based connections, etc. As described further herein, a user may provide input to the input apparatus 142, 162 to view and/or select one or more pieces of information related to the determination of whether a patient may benefit from cardiac conduction system pacing or other cardiac therapies and/or related to the configuration of cardiac therapy delivered by cardiac therapy apparatus such as, e.g., an implantable medical device.

Although as depicted the input apparatus 142 is a keyboard and the input apparatus 162 is a touchscreen, it is to be understood that the input apparatus 142, 162 may include any apparatus capable of providing input to the local computing device 140 and the computing device 160 to perform the functionality, methods, and/or logic described herein. For example, the input apparatus 142, 162 may include a keyboard, a mouse, a trackball, a touchscreen (e.g., capacitive touchscreen, a resistive touchscreen, a multi-touch touchscreen, etc.), etc. Likewise, the display apparatus 130, 170 may include any apparatus capable of displaying information to a user, such as a graphical user interface 132, 172 including one or more metrics indicative of cardiac conduction system therapy benefit, one or more metrics indicative of conventional cardiac pacing therapy benefit, express indications of cardiac conduction system therapy benefit, express indications of conventional cardiac pacing therapy benefit, electrode status information, graphical maps of electrical activation, a plurality of signals for the external electrodes over one or more heartbeats, QRS complexes, various pacing parameters, electrical heterogeneity information (EHI), textual instructions, graphical depictions of anatomy of a human heart, images or graphical depictions of the patient's heart, graphical depictions of locations of one or more electrodes, graphical depictions of a human torso, images or graphical depictions of the patient's torso, graphical depictions or actual images of implanted electrodes and/or leads, etc. Further, the display apparatus 130, 170 may include a liquid crystal display, an organic light-emitting diode screen, a touchscreen, a cathode ray tube display, etc.

The processing programs or routines stored and/or executed by the local computing device 140, the remote computing device 160, and the cloud computing device 190 may include programs or routines for computational mathematics, matrix mathematics, decomposition algorithms, compression algorithms (e.g., data compression algorithms), calibration algorithms, image construction algorithms, signal processing algorithms (e.g., various filtering algorithms, Fourier transforms, fast Fourier transforms, etc.), standardization algorithms, comparison algorithms, vector mathematics, or any other processing used to implement one or more illustrative methods and/or processes described herein. Data stored and/or used by the local computing device 140, the remote computing device 160, and the cloud computing device 190 may include, for example, electrical signal/waveform data from the electrode apparatus 110 (e.g., a plurality of QRS complexes), electrical activation times from the electrode apparatus 110, cardiac sound/signal/waveform data from acoustic sensors, graphics (e.g., graphical elements, icons, buttons, windows, dialogs, pull-down menus, graphic areas, graphic regions, 3D graphics, etc.), graphical user interfaces, results from one or more processing programs or routines employed according to the disclosure herein (e.g., electrical signals, electrical heterogeneity information, etc.), or any other data that may be used for carrying out the one and/or more processes or methods described herein.

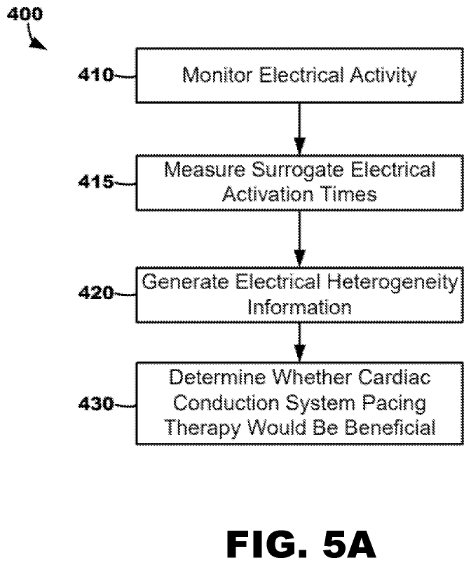

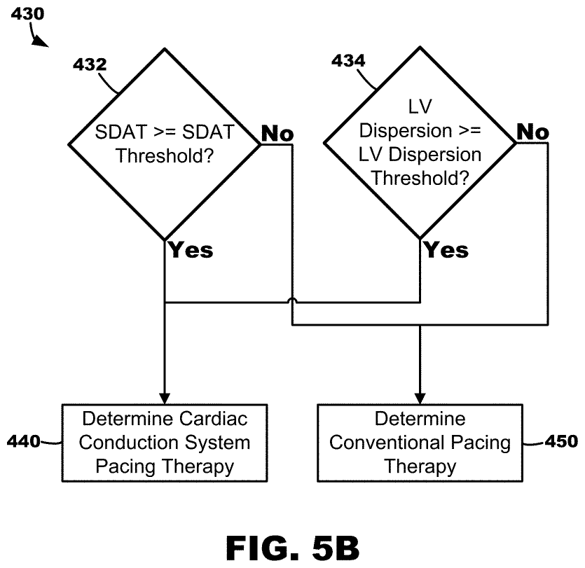

In one or more embodiments, one or more of the local computing device 140, the remote computing device 160, and the cloud computing device 190 may be configured to obtain external electrical activity measured from tissue of a patient during intrinsic activation of the patient's heart (e.g., using the electrode apparatus 110), generate electrical heterogeneity information (EHI) based on the obtained electrical activity such as, e.g., one or more metrics of dispersion of cardiac electrical activation times, and determine that (e.g., determine whether) cardiac conduction system pacing therapy would benefit the patient based on at least the one or more metrics of dispersion of cardiac electrical activation times as will be described further herein with respect to FIGS. 5A-5B. In particular, for example, one or more of the local computing device 140, the remote computing device 160, and the cloud computing device 190 may generate a standard deviation of activation times (SDAT) of the obtained external electrical activity during intrinsic activation and then compare the SDAT to a SDAT threshold such as, e.g., 40 milliseconds. If the generated SDAT is greater than or equal to the SDAT threshold, then one or more of the local computing device 140, the remote computing device 160, and the cloud computing device 190 may determine that the patient would benefit from cardiac conduction system pacing. If the generated SDAT is less than the SDAT threshold, then one or more of the local computing device 140, the remote computing device 160, and the cloud computing device 190 may determine that the patient would benefit from conventional myocardial cardiac pacing therapy.

Further, for example, one or more of the local computing device 140, the remote computing device 160, and the cloud computing device 190 may generate a left-sided metric of dispersion of the obtained external electrical activity from the left side of the patient's torso. The left-sided metric of dispersion may be a left-sided standard deviation of activation times (LVED) of the obtained external electrical activity from the left side of the patient's torso. One or more of the local computing device 140, the remote computing device 160, and the cloud computing device 190 may then compare the left-sided metric of dispersion to a left-sided dispersion threshold such as, e.g., 30 milliseconds. If the generated LVED is greater than or equal to the left-sided dispersion threshold, then one or more of the local computing device 140, the remote computing device 160, and the cloud computing device 190 may determine that the patient would benefit from cardiac conduction system pacing. If the generated LVED is less than the left-sided dispersion threshold, then one or more of the local computing device 140, the remote computing device 160, and the cloud computing device 190 may determine that the patient would benefit from conventional myocardial cardiac pacing therapy.

In one or more embodiments, the illustrative systems, methods, devices, and interfaces may be implemented using one or more computer programs executed on programmable computers, such as computers that include, for example, processing capabilities, data storage (e.g., volatile or non-volatile memory and/or storage elements), input devices, and output devices. Program code and/or logic described herein may be applied to input data to perform functionality described herein and generate desired output information. The output information may be applied as input to one or more other systems, methods, devices, and/or interfaces as described herein or as would be applied in a known fashion.

The one or more programs used to implement the systems, methods, devices, and/or interfaces described herein may be provided using any programmable language, e.g., a high-level procedural and/or object orientated programming language that is suitable for communicating with a computer. Any such programs may, for example, be stored on any suitable device, e.g., a storage media, that is readable by a general or special purpose program running on a computer (e.g., including processing circuitry or processing apparatus) for configuring and operating the computer when the suitable device is read for performing the procedures described herein. In other words, at least in one embodiment, the illustrative systems, methods, devices, and interfaces may be implemented using a tangible computer readable storage medium, configured with a computer program, where the storage medium so configured causes the computer to operate in a specific and predefined manner to perform functions described herein. Further, in at least one embodiment, the illustrative systems, methods, devices, and interfaces may be described as being implemented by logic (e.g., object code) encoded in one or more non-transitory media that includes code for execution and, when executed by a processor or processing circuitry, is operable to perform operations such as the methods, processes, and/or functionality described herein.

The local computing device 140, the remote computing device 160, and the cloud computing device 190 may be, for example, any fixed or mobile computer system (e.g., a controller, a microcontroller, a personal computer, minicomputer, tablet computer, etc.) including processing circuitry. The exact configurations of the local computing device 140, the remote computing device 160, and the cloud computing device 190 are not limiting, and essentially any device including processing circuitry and capable of providing suitable computing capabilities and control capabilities (e.g., signal analysis, mathematical functions such as medians, modes, averages, maximum value determination, minimum value determination, slope determination, minimum slope determination, maximum slope determination, graphics processing, etc.) may be used. As described herein, a digital file may be any medium (e.g., volatile or non-volatile memory, a CD-ROM, a punch card, magnetic recordable tape, etc.) containing digital bits (e.g., encoded in binary, trinary, etc.) that may be readable and/or writeable by the local computing device 140, the remote computing device 160, and the cloud computing device 190 described herein. Also, as described herein, a file in user-readable format may be any representation of data (e.g., ASCII text, binary numbers, hexadecimal numbers, decimal numbers, graphically, etc.) presentable on any medium (e.g., paper, a display, etc.) readable and/or understandable by a user.

In view of the above, it will be readily apparent that the functionality as described in one or more embodiments according to the present disclosure may be implemented in any manner as would be known to one skilled in the art. As such, the computer language, the computer system, or any other software/hardware which is to be used to implement the processes described herein shall not be limiting on the scope of the systems, processes, or programs (e.g., the functionality provided by such systems, processes, or programs) described herein.

The illustrative electrode apparatus 110 may be configured to measure body-surface potentials of a patient 14 and, more particularly, torso-surface potentials of a patient 14. As shown in FIG. 2, the illustrative electrode apparatus 110 may include a set, or array, of external electrodes 112, a strap 113, and interface/amplifier circuitry 116. The electrodes 112 may be attached, or coupled, to the strap 113 and the strap 113 may be configured to be wrapped around the torso of a patient 14 such that the electrodes 112 surround the patient's heart. As further illustrated, the electrodes 112 may be positioned around the circumference of a patient 14, including the posterior, posterolateral, lateral, anterolateral, and anterior locations of the torso of a patient 14.

The illustrative electrode apparatus 110 may be further configured to measure, or monitor, sounds from the patient 14. As shown in FIG. 2, the illustrative electrode apparatus 110 may include a set, or array, of acoustic sensors 120 attached, or coupled, to the strap 113. The strap 113 may be configured to be wrapped around the torso of a patient 14 such that the acoustic sensors 120 surround the patient's heart. As further illustrated, the acoustic sensors 120 may be positioned around the circumference of a patient 14, including the posterior, posterolateral, lateral, anterolateral, and anterior locations of the torso of a patient 14.

Further, the electrodes 112 and the acoustic sensors 120 may be electrically connected to interface/amplifier circuitry 116 via wired connection 118. The interface/amplifier circuitry 116 may be configured to amplify the signals from the electrodes 112 and the acoustic sensors 120 and provide the signals to one or more of the local computing device 140, the remote computing device 160, and the cloud computing device 190. Other illustrative systems may use a wireless connection to transmit the signals sensed by electrodes 112 and the acoustic sensors 120 to the interface/amplifier circuitry 116 and, in turn, to one or more of the local computing device 140, the remote computing device 160, and the cloud computing device 190, e.g., as channels of data. In one or more embodiments, the interface/amplifier circuitry 116 may be electrically coupled to the local computing device 140 using, e.g., analog electrical connections, digital electrical connections, wireless connections, bus-based connections, network-based connections, internet-based connections, etc.

Although in the example of FIG. 2 the electrode apparatus 110 includes a strap 113, any of a variety of mechanisms, e.g., tape or adhesives, may be employed to aid in the spacing and placement of electrodes 112 and the acoustic sensors 120. In some examples, the strap 113 may include an elastic band, strip of tape, or cloth. Further, in some examples, the strap 113 may be part of, or integrated with, a piece of clothing such as, e.g., a t-shirt or hospital gown. In other examples, the electrodes 112 and the acoustic sensors 120 may be placed individually on the torso of a patient 14. Further, in other examples, one or both of the electrodes 112 (e.g., arranged in an array) and the acoustic sensors 120 (e.g., also arranged in an array) may be part of, or located within, patches, vests, and/or other manners of securing the electrodes 112 and the acoustic sensors 120 to the torso of the patient 14. Still further, in other examples, one or both of the electrodes 112 and the acoustic sensors 120 may be part of, or located within, two sections of material or two patches. One of the two patches may be located on the anterior side of the torso of the patient 14 (to, e.g., monitor electrical signals representative of the anterior side of the patient's heart, measure surrogate cardiac electrical activation times representative of the anterior side of the patient's heart, monitor or measure sounds of the anterior side of the patient, etc.) and the other patch may be located on the posterior side of the torso of the patient 14 (to, e.g., monitor electrical signals representative of the posterior side of the patient's heart, measure surrogate cardiac electrical activation times representative of the posterior side of the patient's heart, monitor or measure sounds of the posterior side of the patient, etc.). And still further, in other examples, one or both of the electrodes 112 and the acoustic sensors 120 may be arranged in a top row and bottom row that extend from the anterior side of the patient 14 across the left side of the patient 14 to the posterior side of the patient 14. Yet still further, in other examples, one or both of the electrodes 112 and the acoustic sensors 120 may be arranged in a curve around the armpit area and may have an electrode/sensor-density that less dense on the right thorax that the other remaining areas.

The electrodes 112 may be configured to surround the heart of the patient 14 and record, or monitor, the electrical signals associated with the depolarization and repolarization of the heart after the signals have propagated through the torso of a patient 14. Each of the electrodes 112 may be used in a unipolar configuration to sense the torso-surface potentials that reflect the cardiac signals. The interface/amplifier circuitry 116 may also be coupled to a return or indifferent electrode (not shown) that may be used in combination with each electrode 112 for unipolar sensing.

In some examples, there may be about 12 to about 40 electrodes 112 and about 12 to about 40 acoustic sensors 120 spatially distributed around the torso of a patient. Other configurations may have more or fewer electrodes 112 and more or fewer acoustic sensors 120. For example, the number of electrodes 112 of the electrode apparatus 110 may be greater than or equal to 10, greater than or equal to 12, greater than or equal to 15, greater than or equal to 20, greater than or equal to 35, greater than or equal to 40, etc. and/or less than or equal to 80 electrodes, less than or equal to 70 electrodes, less than or equal to 60 electrodes, less than or equal to 50 electrodes, less than or equal to 45 electrodes, less than or equal to 38 electrodes, etc. In at least one embodiment, the electrode apparatus 110 includes 40 electrodes 112 with 20 of the electrodes 112 configured to be located on the posterior of the patient and the 20 of the electrodes 112 configured to be located on the anterior of the patient. In some embodiments, more electrodes 112 may be configured to be positioned on the anterior of the patient than the posterior of the patient or more electrodes 112 may be configured to be positioned on the posterior of the patient than the anterior of the patient. For example, in one embodiment, 25 electrodes 112 may be configured to be positioned on the anterior of the patient and 15 electrodes 112 may be configured to be positioned on the posterior of the patient. Additionally, in one embodiment, the electrodes 112 may be included as part of a 12-lead ECG apparatus. It is to be understood that the electrodes 112 and acoustic sensors 120 may not be arranged or distributed in an array extending all the way around or completely around the patient 14. Instead, the electrodes 112 and acoustic sensors 120 may be arranged in an array that extends only part of the way or partially around the patient 14. For example, the electrodes 112 and acoustic sensors 120 may be distributed on the anterior, posterior, and left sides of the patient with less or no electrodes and acoustic sensors proximate the right side (including posterior and anterior regions of the right side of the patient).

One or more of the local computing device 140, the remote computing device 160, and the cloud computing device 190 may record and analyze the torso-surface potential signals sensed by electrodes 112 and the sound signals sensed by the acoustic sensors 120, which are amplified/conditioned by the interface/amplifier circuitry 116. One or more of the local computing device 140, the remote computing device 160, and the cloud computing device 190 may be configured to analyze the electrical signals from the electrodes 112 to provide electrocardiogram (ECG) signals, information, or data from the patient's heart as will be further described herein. One or more of the local computing device 140, the remote computing device 160, and the cloud computing device 190 may be configured to analyze the electrical signals from the acoustic sensors 120 to provide sound signals, information, or data from the patient's body and/or devices implanted therein.

Additionally, the local computing device 140, the remote computing device 160, and the cloud computing device 190 may be configured to provide graphical user interfaces 132, 172 depicting various information related to the electrode apparatus 110 and the data gathered, or sensed, using the electrode apparatus 110. In at least one embodiment, the cloud computing device 190 may provide one or both of the graphical user interfaces 132, 172 to the local computing device 140 and the remote computing device 160. For example, the graphical user interfaces 132, 172 may depict cardiac electrical activation maps obtained using the electrode apparatus 110 and sound data including sound waves obtained using the acoustic sensors 120 as well as other information related thereto. Illustrative systems, methods, devices, and interfaces may noninvasively use the electrical information collected using the electrode apparatus 110 and the sound information collected using the acoustic sensors 120 to evaluate, or determine, whether a patient would be benefit from cardiac conduction system pacing or conventional cardiac pacing therapies.

Further, the electrode apparatus 110 may further include reference electrodes and/or drive electrodes to be, e.g., positioned about the lower torso of the patient 14, that may be further used by the system 100. For example, the electrode apparatus 110 may include three reference electrodes, and the signals from the three reference electrodes may be combined to provide a reference signal. Further, the electrode apparatus 110 may use three caudal reference electrodes (e.g., instead of standard references used in a Wilson Central Terminal) to get a “true” unipolar signal with less noise from averaging three caudally located reference signals.

FIG. 3 illustrates another illustrative electrode apparatus 110 that includes a plurality of electrodes 112 configured to surround the heart of the patient 14 and record, or monitor, the electrical signals associated with the depolarization and repolarization of the heart after the signals have propagated through the torso of the patient 14 and a plurality of acoustic sensors 120 configured to surround the heart of the patient 14 and record, or monitor, the sound signals associated with the heart after the signals have propagated through the torso of the patient 14. The electrode apparatus 110 may include a vest 114 upon which the plurality of electrodes 112 and the plurality of acoustic sensors 120 may be attached, or to which the electrodes 112 and the acoustic sensors 120 may be coupled. In at least one embodiment, the plurality, or array, of electrodes 112 may be used to collect electrical information such as, e.g., surrogate electrical activation times. Similar to the electrode apparatus 110 of FIG. 2, the electrode apparatus 110 of FIG. 3 may include interface/amplifier circuitry 116 electrically coupled to each of the electrodes 112 and the acoustic sensors 120 through a wired connection 118 and be configured to transmit signals from the electrodes 112 and the acoustic sensors 120 to computing apparatus such as the local computing device 140. As illustrated, the electrodes 112 and the acoustic sensors 120 may be distributed over the torso of a patient 14, including, for example, the posterior, posterolateral, lateral, anterolateral, and anterior locations of the torso of a patient 14.

The vest 114 may be formed of fabric with the electrodes 112 and the acoustic sensors 120 attached to the fabric. The vest 114 may be configured to maintain the position and spacing of electrodes 112 and the acoustic sensors 120 on the torso of the patient 14. Further, the vest 114 may be marked to assist in determining the location of the electrodes 112 and the acoustic sensors 120 on the surface of the torso of the patient 14. In some examples, there may be about 25 to about 256 electrodes 112 and about 25 to about 256 acoustic sensors 120 distributed around the torso of the patient 14, though other configurations may have more or fewer electrodes 112 and more or fewer acoustic sensors 120.

The illustrative systems, methods, devices, and interfaces may be used to provide noninvasive assistance to a user in the evaluation of a patient's cardiac health and determine whether the patient may benefit from cardiac conduction system pacing or conventional pacing therapies (e.g., left ventricular myocardial pacing therapy). For example, the illustrative systems, methods, devices, and interfaces may be used to obtain (e.g., measure, monitor, receive from other electrode apparatus) electrical activity of the patient and generated electrical heterogeneity information therefrom, and in particular, metrics of dispersion of cardiac electrical activation times, which may then be used to determine whether the patient may benefit from cardiac conduction system pacing. Further, it is to be understood that the computing apparatus, e.g., include the local computing device 140, the remote computing device 160, and the cloud computing device 190, may be operatively coupled in a plurality of different ways so as to perform, or execute, the functionality described herein. For example, in the embodiment depicted, the computing device 140 may be wireless operably coupled to the remote computing device 160 as depicted by the wireless signal lines emanating therebetween. Additionally, as opposed to wireless connections, one or more of the local computing device 140 and the remoting computing device 160 may be operably coupled through one or wired electrical connections. Furthermore, one or both of the local computing device 140 and the remote computing device 160 may be operably coupled (e.g., wirelessly operably coupled, partially-wirelessly coupled, etc.) to the cloud computing device 190 via a network 191 such as, e.g., the internet. In one embodiment, the cloud computing device 190 may perform all or much of the data analysis on the data from the electrode apparatus 110 and output such analysis to one or both of the local computing device 140 and the remote computing device 160. It is be understood that the data processing and analysis described herein may be provide by one or more of the local computing device 140, the remote computing device 160, and the cloud computing device 190. In other words, the processing and analysis may be distributed among the computing apparatus such as the local computing device 140, the remote computing device 160, and the cloud computing device 190. Additionally, the cloud computing device 190 may include, among other things, an electronic medical records (EMR) system or database that may store information from the systems, methods, devices, apparatus, and interfaces described herein and provide information to such systems, methods, devices, apparatus, and interfaces. For example, an EMR of the cloud computing device 190 may provide images (e.g., CT images of the patient's heart) to local computing device 140 and the remote computing device 160 to be used with the data provided by the electrode apparatus 110.

The illustrative systems, devices, methods, and interfaces described herein may provide users (e.g., clinicians, doctors, etc.) a useful tool to determine whether a patient would benefit from cardiac conduction system pacing therapy and/or another cardiac therapies. Further the illustrative systems, devices, methods, and interfaces described herein may provide users a useful tool to determine whether intrinsic heterogeneity and/or asynchrony in electrical activation may be corrected through conduction system pacing or if traditional myocardial pacing (e.g., a left ventricular lead in the coronary sinus delivering pacing to myocardial tissue on the free wall) may be more appropriate based on evaluation of patient's intrinsic activation before implanting any pacing lead. This may be helpful for pre-procedural planning in selecting the type of lead (e.g., a conduction system pacing lead versus a traditional left ventricular pacing lead) and device (e.g., dual chamber versus triple chamber).

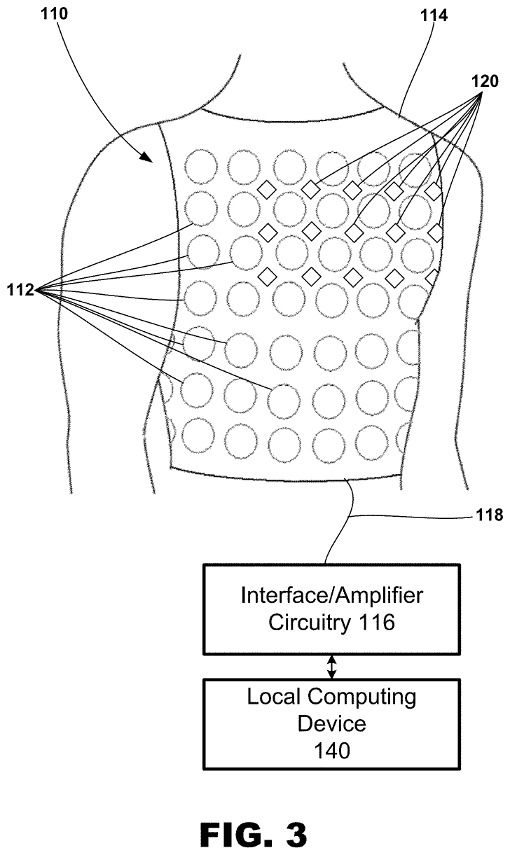

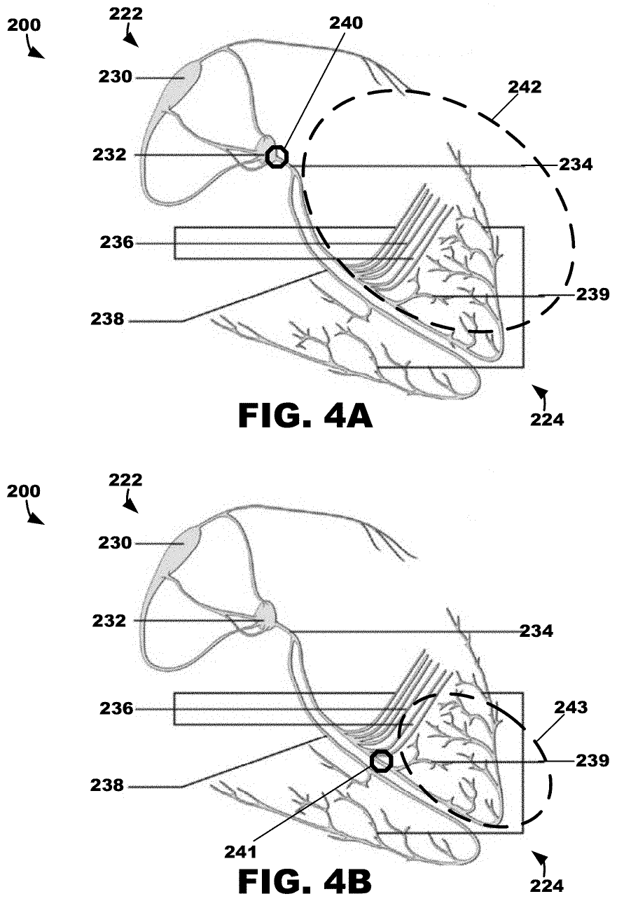

A patient's cardiac conduction network 200 is depicted in FIGS. 4A-4B. As shown, the cardiac conduction network 200 extends from a proximal region 222 to a distal region 224. The cardiac conduction network 200 includes a specialized network of cells comprising the left and right bundle branches as well as a highly branched network of specialized Purkinje fibers that aids in rapid propagation of electrical activation across the ventricles, which may lead to a very synchronized activation of the heart. The cardiac conduction system is part of the natural pathway of electrical conduction that extends from the sinoatrial node 230 to the ventricles via the atrioventricular node 232. Further, the electrical impulses that trigger depolarization of the myocardial tissue of the patient's heart to effectively “beat” traverse the cardiac conduction network 200 from the sinoatrial node 230 to the Purkinje fibers 239.

As described, herein, the proximal region 222 of the cardiac conduction network 200 may include the sinoatrial node 230 and the atrioventricular node 232 and the intermodal pathways therebetween, and the distal region 224 of the cardiac conduction network 200 may include the right bundle branch 238, the left posterior bundle 236, and the Purkinje fibers 239. In particular, the most distal area of the cardiac conduction network 200 may be the ends of the Purkinje fibers 239 and the most proximal area of the cardiac conduction network 200 may be the sinoatrial node 230. Thus, the cardiac conduction network 200 may be described as extending from the sinoatrial node 230 to the Purkinje fibers 239.

In FIG. 4A, a cardiac conduction system block 240 is positioned just distal of the atrioventricular node 232 but prior to the bundle of His 234 branching to the left and right bundles. Thus, it may be described that the cardiac conduction system block 240 is positioned relatively proximally along the cardiac conduction network 220. Using the illustrative systems, methods, devices, and interfaces as described further herein, electrical heterogeneity information including one or more metrics of dispersion of cardiac electrical activation times may be determined that indicate a large extent 242 of delay in the left ventricle when a cardiac conduction system block 240 is positioned as shown in FIG. 4A. Thus, cardiac conduction system block 240 may be a good candidate for cardiac conduction system pacing therapy because, e.g., cardiac conduction system pacing therapy may be delivered to a position, or location, within the cardiac conduction system distal of the cardiac conduction system block 240. For example, cardiac conduction system pacing therapy may be delivered to the bundle of His 234 and/or one of both of the right and left branches.

In FIG. 4B, a cardiac conduction system block 241 is positioned along the left branch just distal of the left posterior bundle. Thus, it may be described that the cardiac conduction system block 241 is positioned relatively distally along the cardiac conduction network 220. Using the illustrative systems, devices, methods, and interfaces as described further herein, electrical heterogeneity information including one or more metrics of dispersion of cardiac electrical activation times may be determined that indicate a small extent 243 of delay in the left ventricle when a cardiac conduction system block 241 is positioned as shown in FIG. 4B. Thus, cardiac conduction system block 241 may not be a good candidate for cardiac conduction system pacing therapy because, e.g., cardiac conduction system pacing therapy likely could not be positioned more distal than the cardiac conduction system block 241, and if the cardiac conduction system pacing therapy were positioned proximal to the cardiac conduction system block 241 (such as, e.g., at the bundle of His 234), any such cardiac conduction system pacing therapy may be blocked, or stopped, by the cardiac conduction system block 241. Therefore, when comparing the cardiac conduction system blocks 240, 241 of FIGS. 4A-4B, the more proximal cardiac conduction system block 240 is likely more correctable using cardiac conduction system pacing therapy than the more distal cardiac conduction system block 241 of FIG. 4B.

An illustrative method 400 for determining whether cardiac conduction system pacing therapy would be beneficial is depicted in FIG. 5A. As shown, the method 400 includes monitoring 410 electrical activity to generate a plurality of electrical signals (e.g., ECG or cardiac signals). The electrical activity may be monitored during intrinsic heart rhythm of the patient without delivery of any cardiac therapy. Thus, the method 400 may be performed prior to the implantation of any implantable cardiac therapy device. For example, the method 400 may be performed during an initial consultation prior to any invasive procedures to treat the present condition. Additionally, as described herein, monitoring electrical activity 410 using a plurality of external electrodes is a noninvasive process since, e.g., the external electrodes are attached to the skin of the patient as opposed to inserting or implanting any electrodes to acquire electrical activity or data. Additionally, however, if an implantable cardiac therapy device is already implanted in the patient, the method 400 may be performed with any cardiac therapy provided by the implantable cardiac therapy device disabled (or “turned off”).

According to various embodiments, the electrical activity is monitored 410 using a plurality of electrodes. The plurality of electrodes may be external surface electrodes configured in a band or a vest similar to as described herein with respect to FIGS. 1-3. Each of the electrodes may be positioned or located about the torso of the patient so as to monitor electrical activity (e.g., acquire torso-potentials) from a plurality of different locations about the torso of the patient. Each of the different locations where the electrodes are located may correspond to the electrical activation of different portions or regions of cardiac tissue of the patient's heart. Thus, for example, the plurality of electrodes may record, or monitor, the electrical signals associated with the depolarization and repolarization of a plurality of different locations of, or about, the heart after the signals have propagated through the torso of a patient. According to various embodiments, the plurality of external electrodes may include, or comprise, a plurality of anterior electrodes that are located proximate skin of the anterior of the patient's torso, left lateral or left-sided electrodes that are located proximate skin of the left lateral or left side of the patient's torso, and posterior electrodes that are located proximate skin of the posterior of the patient's torso.

It may be described that, when using a plurality of external electrodes, the monitoring process 410 may provide a plurality of electrocardiograms (ECGs), which are signals representative of the depolarization and repolarization of the patient's heart. The plurality of ECGs may, in turn, be used to generate surrogate cardiac electrical activation times 415 representative of the depolarization of the myocardial tissue of the heart. As described herein, surrogate cardiac electrical activation times may be, for example, representative of actual, or local, electrical activation times of one or more regions of the patient's heart. Measurement of activation times can be performed by picking an appropriate fiducial point (e.g., peak values, minimum values, minimum slopes, maximum slopes, zero crossings, threshold crossings, etc. of a near or far-field EGM) and measuring time between the onset of cardiac depolarization (e.g., earliest onset of all QRS complexes) and the appropriate fiducial point (e.g., within the electrical activity). In at least one embodiment, the earliest QRS onset from all of the plurality of electrodes may be utilized as the starting point for each activation time for each electrode, and the maximum slope following the onset of the QRS complex may be utilized as the end point of each activation time for each electrode.

The monitored electrical activity 410 and, in turn, the electrical activation times 415 may be used to generate electrical heterogeneity information (EHI) 420. The EHI (e.g., data) may be defined as information indicative of at least one of mechanical synchrony or dyssynchrony of the heart and/or electrical synchrony or dyssynchrony of the heart. In other words, EHI may represent a surrogate of actual mechanical and/or electrical functionality of a patient's heart. In at least one embodiment, the EHI may be used to determine a surrogate value representative of hemodynamic response (e.g., LV pressure gradients). Left ventricular pressure may be typically monitored invasively with a pressure sensor located in the left ventricular of a patient's heart. As such, the use of EHI to determine a surrogate value representative of the left ventricular pressure may avoid invasive monitoring using a left ventricular pressure sensor.

In one or more embodiments, the EHI may include one or more metrics or indices of dispersion of the surrogate electrical activities and/or monitored electrical activity. The one or more metrics of dispersion of cardiac electrical activation times may include a standard deviation of activation times measured using some or all of the external electrodes, e.g., of the electrode apparatus 110 described herein with respect FIGS. 1-3. Further, local, or regional, EHI may include standard deviations and/or averages of activation times measured using electrodes located in certain anatomic areas of the torso. For example, external electrodes on the left side of the torso of a patient may be used to compute local, or regional, left EHI. In particular, for example, the one or more metrics of dispersion of cardiac electrical activation times may include one or both of a standard deviation of activation times (SDAT) of the obtained external electrical activity from all of the plurality of electrodes positioned about the patient's toros and a left-sided standard deviation of activation times (LVED) of the obtained external electrical activity from some of the plurality of electrodes positioned about the left side of the patient's torso.

The EHI may be generated using one or more various systems and/or methods. For example, EHI may be generated using an array, or a plurality, of surface electrodes and/or imaging systems as described in U.S. Pat. No. 9,510,763 B2 issued on Dec. 6, 2016, and entitled “ASSESSING INRA-CARDIAC ACTIVATION PATTERNS AND ELECTRICAL DYSSYNCHRONY,” U.S. Pat. No. 8,972,228 B2 issued Mar. 3, 2015, and entitled “ASSESSING INTRA-CARDIAC ACTIVATION PATTERNS”, and U.S. Pat. No. 8,180,428 B2 issued May 15, 2012 and entitled “METHODS AND SYSTEMS FOR USE IN SELECTING CARDIAC PACING SITES,” each of which is incorporated herein by reference in its entirety.

As described herein, the EHI may include one or more left, or left-sided, metrics, such as LVED, generated based on left-sided activation times of the surrogate cardiac electrical activation times measured using a plurality of left external electrodes. The left external electrodes may include a plurality of left external electrodes positioned to the left side of the patient's torso.

Another left, or left-sided metric, or index, of electrical heterogeneity may include an average of surrogate cardiac electrical activation times (LVAT) monitored by external electrodes located proximate the left side of a patient. The LVED and LVAT may be determined (e.g., calculated, computed, etc.) from electrical activity measured only by electrodes proximate the left side of the patient, which may be referred to as “left” electrodes. Activation time determined, or measured, from the left electrodes may be described as being left-sided activation times. The left electrodes may be defined as any surface electrodes located proximate the left ventricle, which includes the body or torso regions to the left of the patient's sternum and spine (e.g., toward the left arm of the patient, the left side of the patient, etc.). In one embodiment, the left electrodes may include all anterior electrodes on the left of the sternum and all posterior electrodes to the left of the spine. In another embodiment, the left electrodes may include all anterior electrodes on the left of the sternum and all posterior electrodes. In yet another embodiment, the left electrodes may be designated based on the contour of the left and right sides of the heart as determined using imaging apparatus (e.g., x-ray, fluoroscopy, etc.).

The illustrative method 400 may then determine whether cardiac conduction system pacing therapy would benefit the patient 430 based on the generated EHI, and in particular, the one or more metrics of dispersion of cardiac electrical activation times. For example, one or both of SDAT and LVED may be analyzed to determine whether cardiac conduction system pacing therapy would benefit the patient 430. Additionally, it is to be understood that determining whether cardiac conduction system pacing therapy would benefit the patient 430 may not necessarily be a binary, or yes-or-no, determination, and instead, may be a likelihood of cardiac conduction system pacing therapy success for the patient. For instance, the likelihood of cardiac conduction system pacing therapy success may be expressed, or represented, by a percentage or through descriptors such as, e.g., “cardiac conduction system pacing therapy highly likely to be beneficial,” “cardiac conduction system pacing therapy likely to be beneficial,” “cardiac conduction system pacing therapy unlikely to be beneficial,” and “cardiac conduction system pacing therapy highly unlikely to be beneficial.”

Additionally, for example, an indication of whether the cardiac conduction system pacing therapy would benefit the patient based on the generated EHI may be displayed on a graphical user interface. More specifically, for instance, after electrode apparatus including a plurality of external electrodes has been applied the patient, a user (e.g., clinician, doctor, etc.) may use a graphical user interface displayed on display apparatus to initiate a cardiac conduction system benefit determination by, e.g., selecting a button or other area on the graphical user interface. Thus, in in response to the user initiating the cardiac conduction system pacing therapy benefit determination, the illustrative systems, devices, and methods may monitor electrical activity 410, measure surrogate cardiac electrical activation times 415, generate EHI 420, determine whether cardiac conduction system pacing therapy would benefit the patient 430 based on the generated EHI, and then display an indication of whether the cardiac conduction system pacing therapy would benefit the patient on the graphical user interface.

An illustrative method 430 of determining whether cardiac conduction system pacing therapy would benefit the patient is shown in FIG. 5B. The illustrative method 430 may utilize one or both of SDAT and LVED to determine whether cardiac conduction system pacing therapy would benefit the patient 430.

The method 430 may include comparing SDAT to a SDAT threshold 432. The SDAT threshold may be between about 25 milliseconds (ms) and 50 ms. In at least one embodiment, the SDAT threshold is 40 ms. The SDAT threshold may be greater than or equal to 25 ms, greater than or equal to 35 ms, greater than or equal to 40 ms, greater than or equal to 45 ms, etc. and/or less than or equal to 65 ms, less than or equal to 60 ms, less than or equal to 55 ms, less than or equal to 50 ms, etc.

If the SDAT generated from the electrical activity monitored during intrinsic activation is greater than or equal to the SDAT threshold, then cardiac conduction system pacing therapy is determined to be beneficial 440. Conversely, if the SDAT generated from the electrical activity monitored during intrinsic activation is less than the SDAT threshold, then conventional cardiac pacing therapy (e.g., myocardial tissue pacing therapy) may be determined to be beneficial 450. Additionally, if the SDAT generated from the electrical activity monitored during intrinsic activation is greater than or equal to the SDAT threshold, then it may be determined that the cardiac conduction system block is determined to be located, or positioned, closer to the proximal region of the cardiac conduction network, and if the SDAT generated from the electrical activity monitored during intrinsic activation is less than the SDAT threshold, then the cardiac conduction system block is determined to be located, or positioned, closer to the distal region of the cardiac conduction network. Thus, in the embodiment where the SDAT threshold is 40 ms, if the SDAT generated from the electrical activity monitored during intrinsic activation is greater than or equal to 40 ms, then cardiac conduction system pacing therapy is determined to be beneficial 440, and if the SDAT generated from the electrical activity monitored during intrinsic activation is less than 40 ms, then conventional cardiac pacing therapy is determined to be beneficial 450.

The method 430 may include comparing LVED to a left-sided dispersion threshold 434. The left-sided dispersion threshold may be between about 25 ms and 40 ms. In at least one embodiment, the left-sided dispersion threshold is 30 ms. The left-sided dispersion threshold may be greater than or equal to 25 ms, greater than or equal to 30 ms, greater than or equal to 35 ms, greater than or equal to 40 ms, etc. and/or less than or equal to 55 ms, less than or equal to 50 ms, less than or equal to 45 ms, etc.

If the LVED generated from the electrical activity monitored during intrinsic activation is greater than or equal to the left-sided dispersion threshold, then cardiac conduction system pacing therapy is determined to be beneficial 440. Conversely, if the LVED generated from the electrical activity monitored during intrinsic activation is less than the left-sided dispersion threshold, then conventional cardiac pacing therapy (e.g., myocardial tissue pacing therapy) may be determined to be beneficial 450. Additionally, if the LVED generated from the electrical activity monitored during intrinsic activation is greater than or equal to the left-sided dispersion threshold, then it may be determined that the cardiac conduction system block is determined to be located, or positioned, closer to the proximal region of the cardiac conduction network, and if the LVED generated from the electrical activity monitored during intrinsic activation is less than the left-sided dispersion threshold, then the cardiac conduction system block is determined to be located, or positioned, closer to the distal region of the cardiac conduction network. Thus, in the embodiment where the left-sided dispersion threshold is 30 ms, if the LVED generated from the electrical activity monitored during intrinsic activation is greater than or equal to 30 ms, then cardiac conduction system pacing therapy is determined to be beneficial 440, and if the LVED generated from the electrical activity monitored during intrinsic activation is less than 30 ms, then conventional cardiac pacing therapy is determined to be beneficial 450.

Optionally, the metric of dispersion determination processes 432, 434 may be used in conjunction to determine whether cardiac conduction system pacing therapy would benefit the patient 430. For example, in such embodiment, both the SDAT must be greater than or equal to the SDAT threshold 432 and the LVED must be greater than or equal to the left-sided dispersion threshold 434 for a determination that the cardiac conduction system pacing therapy is beneficial 440. Additionally, in this embodiment, if only one of the SDAT and LVED are greater than or equal to their respective threshold, then conventional cardiac pacing therapy is determined to be beneficial.

As described herein, the illustrative systems and methods may assist a user (e.g., clinician, doctor, etc.) to determine whether a patient may benefit from cardiac conduction system pacing therapy and/or determine the location of cardiac conduction system block within or along the cardiac conduction network. In one or more embodiments, illustrative cardiac conduction system pacing therapy may utilize any implantable or non-implantable cardiac pacing system intended to pace or deliver electrical paces to one or more areas or regions of the cardiac conduction system of the patient. The cardiac conduction system pacing therapy may use a single pacing electrode defining a single pacing vector or a plurality of pacing electrodes defining a plurality of pacing vectors.

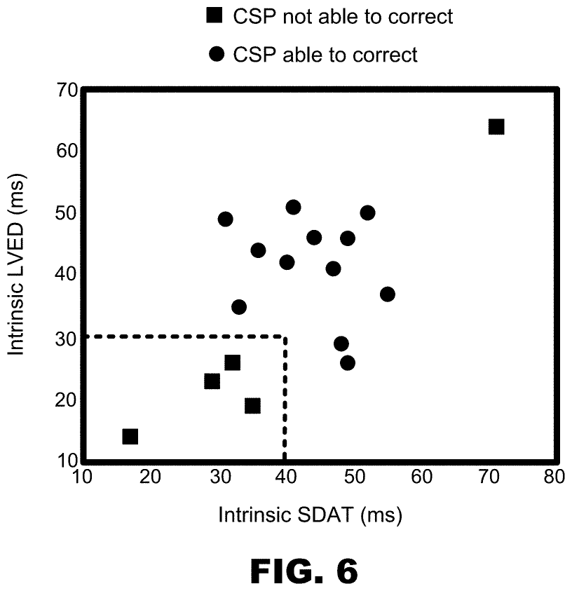

A scatterplot of the standard deviation of activation times and left-sided standard deviation of activation times for a plurality of patients is depicted in FIG. 6. In particular, a clinical study was performed where SDAT and LVED were measured from 17 patients during intrinsic activation, and subsequently, cardiac conduction system pacing (e.g., His bundle and/or left bundle branch pacing) was delivered to such patients. Cardiac conduction system pacing therapy was able to correct 12 of the patients (e.g., improve the cardiac functionality of the patient's heart), each of which patient is represented by circles on the scatter plot. Cardiac conduction system pacing therapy was not able to correct 5 (e.g., not improve the cardiac functionality of the patient's heart) of the patients, each of which patient is represented by squares on the scatter plot. As shown, 4 out 5 (80%) patients whose cardiac functionality was not able to be corrected by cardiac conduction system pacing had a lower intrinsic SDAT (e.g., less than 40 ms as depicted by the dotted line) and had a lower intrinsic LVED (e.g., less than 30 ms as depicted by the dotted line).

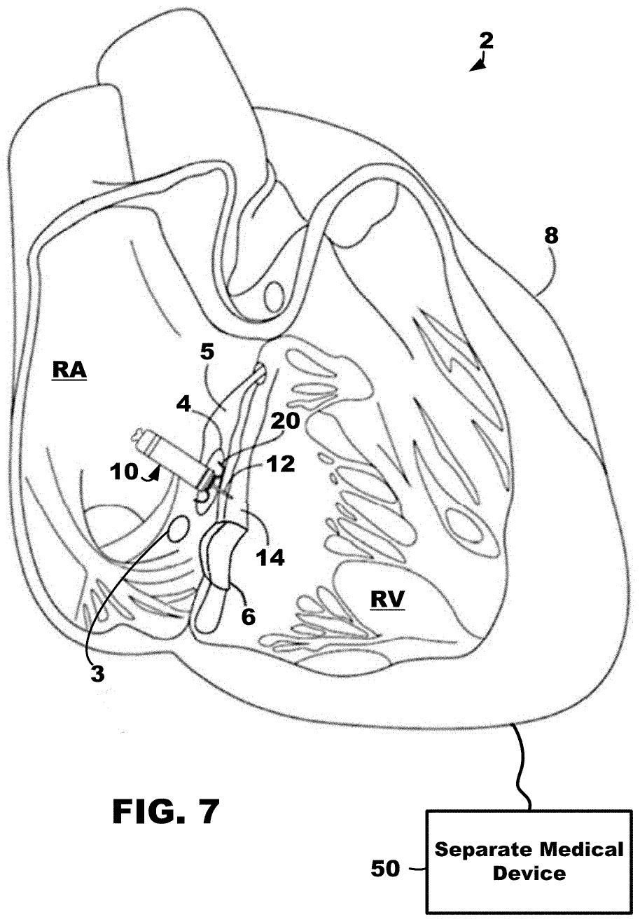

One example of cardiac conduction system pacing therapy may be ventricle from atrium (VfA) pacing therapy described and shown herein with respect to FIGS. 7-10. The VfA pacing therapy may also deliver electrical paces to one or more areas of the cardiac conduction system including, but not limited to areas of the left bundle branches and the right bundle branches. Additionally, the VfA pacing therapy may be configured to deliver electrical paces to myocardial tissue of the patient's left ventricle. Another example of cardiac conduction system pacing therapy may be His bundle pacing therapy as, e.g., described in U.S. Pat. App. Pub. No. 2019/0111270 Al published on Apr. 18, 2019, entitled “His Bundle and Bundle Branch Pacing Adjustment,” which is incorporated herein by reference in its entirety. Still another example of cardiac conduction system pacing therapy may be intraseptal left ventricular endocardial pacing therapy as, e.g., described in U.S. Pat. No. 7,177,704 issued on Feb. 13, 2007, entitled “Pacing Method and Apparatus,” which is incorporated herein by reference in its entirety.

An illustrative ventricle from atrium (VfA) cardiac therapy system is depicted in FIG. 7 that may be configured to be used with, for example, the systems, methods, devices, and interfaces described herein with respect to FIGS. 1-6. Although it is to be understood that the present disclosure may utilize one or both of leadless and leaded implantable medical devices, the illustrative cardiac therapy system of FIG. 7 includes a leadless intracardiac medical device 10 that may be configured for single or dual chamber therapy and implanted in a patient's heart 8. In some embodiments, the device 10 may be configured for single chamber pacing and may, for example, switch between single chamber and multiple chamber pacing (e.g., dual or triple chamber pacing). As used herein, “intracardiac” refers to a device configured to be implanted entirely within a patient's heart, for example, to provide cardiac therapy. The device 10 is shown implanted in the right atrium (RA) of the patient's heart 8 in a target implant region 4. The device 10 may include one or more fixation members 20 that anchor a distal end of the device 10 against the atrial endocardium in a target implant region 4. The target implant region 4 may lie between the Bundle of His 5 and the coronary sinus 3 and may be adjacent, or next to, the tricuspid valve 6. The device 10 may be described as a ventricle-from-atrium device because, for example, the device 10 may perform, or execute, one or both of sensing electrical activity from and providing therapy to one or both ventricles (e.g., right ventricle, left ventricle, or both ventricles, depending on the circumstances) while being generally disposed in the right atrium. In particular, the device 10 may include a tissue-piercing electrode that may be implanted in the basal and/or septal region of the left ventricular myocardium of the patient's heart from the triangle of Koch region of the right atrium through the right atrial endocardium and central fibrous body. Additionally, the tissue-piercing electrode that may be implanted in the basal and/or septal region of the left ventricular myocardium of the patient's heart from the triangle of Koch region of the right atrium through the right atrial endocardium and central fibrous body so as to deliver pacing pulses, or paces, to one or more to one or more areas of the cardiac conduction system including, but not limited to areas of the left bundle branches and the right bundle branches.

The device 10 may be described as a leadless implantable medical device. As used herein, “leadless” refers to a device being free of a lead extending out of the patient's heart 8. Further, although a leadless device may have a lead, the lead would not extend from outside of the patient's heart to inside of the patient's heart or would not extend from inside of the patient's heart to outside of the patient's heart. Some leadless devices may be introduced through a vein, but once implanted, the device is free of, or may not include, any transvenous lead and may be configured to provide cardiac therapy without using any transvenous lead. Further, a leadless VfA device, in particular, does not use a lead to operably connect to an electrode in the ventricle when a housing of the device is positioned in the atrium. Additionally, a leadless electrode may be coupled to the housing of the medical device without using a lead between the electrode and the housing.

The device 10 may include a dart electrode assembly 12 defining, or having, a straight shaft extending from a distal end region of device 10. The dart electrode assembly 12 may be placed, or at least configured to be placed, through the atrial myocardium and the central fibrous body and into the ventricular myocardium 14, or along the ventricular septum, without perforating entirely through the ventricular endocardial or epicardial surfaces. The dart electrode assembly 12 may carry, or include, an electrode at a distal end region of the shaft such that the electrode may be positioned within the ventricular myocardium for sensing ventricular signals and delivering ventricular pacing pulses (e.g., to depolarize the left ventricle and/or right ventricle to initiate a contraction of the left ventricle and/or right ventricle). The ventricular pulses may be delivered directly to the myocardial tissue of the patient's heart and may also be delivered directly to one or more areas of the cardiac conduction system including, but not limited to areas of the left bundle branches and the right bundle branches. In some examples, the electrode at the distal end region of the shaft is a cathode electrode provided for use in a bipolar electrode pair for pacing and sensing. While the implant region 4 as illustrated may enable one or more electrodes of the dart electrode assembly 12 to be positioned in the ventricular myocardium, it is recognized that a device having the aspects disclosed herein may be implanted at other locations for multiple chamber pacing (e.g., dual or triple chamber pacing), single chamber pacing with multiple chamber sensing, single chamber pacing and/or sensing, or other clinical therapy and applications as appropriate.

It is to be understood that although device 10 is described herein as including a single dart electrode assembly, the device 10 may include more than one dart electrode assembly placed, or configured to be placed, through the atrial myocardium and the central fibrous body, and into the ventricular myocardium 14, or along the ventricular septum, without perforating entirely through the ventricular endocardial or epicardial surfaces. Additionally, each dart electrode assembly may carry, or include, more than a single electrode at the distal end region, or along other regions (e.g., proximal or central regions), of the shaft.

The cardiac therapy system 2 may also include a separate medical device 50 (depicted diagrammatically in FIG. 7), which may be positioned outside the patient's heart 8 (e.g., subcutaneously) and may be operably coupled to the patient's heart 8 to deliver cardiac therapy thereto. In one example, separate medical device 50 may be an extravascular ICD. In some embodiments, an extravascular ICD may include a defibrillation lead including, or carrying, a defibrillation electrode. A therapy vector may exist between the defibrillation electrode on the defibrillation lead and a housing electrode of the ICD. Further, one or more electrodes of the ICD may also be used for sensing electrical signals related to the patient's heart 8. The ICD may be configured to deliver shock therapy including one or more defibrillation or cardioversion shocks. For example, if an arrhythmia is sensed, the ICD may send a pulse via the electrical lead wires to shock the heart and restore its normal rhythm. In some examples, the ICD may deliver shock therapy without placing electrical lead wires within the heart or attaching electrical wires directly to the heart (subcutaneous ICDs). Examples of extravascular, subcutaneous ICDs that may be used with the system 2 described herein may be described in U.S. Pat. No. 9,278,229 (Reinke et al.), issued 8 Mar. 2016, which is incorporated herein by reference in its entirety.

In the case of shock therapy (e.g., defibrillation shocks provided by the defibrillation electrode of the defibrillation lead), the separate medical device 50 (e.g., extravascular ICD) may include a control circuit that uses a therapy delivery circuit to generate defibrillation shocks having any of a number of waveform properties, including leading-edge voltage, tilt, delivered energy, pulse phases, and the like. The therapy delivery circuit may, for instance, generate monophasic, biphasic, or multiphasic waveforms. Additionally, the therapy delivery circuit may generate defibrillation waveforms having different amounts of energy. For example, the therapy delivery circuit may generate defibrillation waveforms that deliver a total of between approximately 60-80 Joules (J) of energy for subcutaneous defibrillation.

The separate medical device 50 may further include a sensing circuit. The sensing circuit may be configured to obtain electrical signals sensed via one or more combinations of electrodes and to process the obtained signals. The components of the sensing circuit may include analog components, digital components, or a combination thereof. The sensing circuit may, for example, include one or more sense amplifiers, filters, rectifiers, threshold detectors, analog-to-digital converters (ADCs), or the like. The sensing circuit may convert the sensed signals to digital form and provide the digital signals to the control circuit for processing and/or analysis. For example, the sensing circuit may amplify signals from sensing electrodes and convert the amplified signals to multi-bit digital signals by an ADC, and then provide the digital signals to the control circuit. In one or more embodiments, the sensing circuit may also compare processed signals to a threshold to detect the existence of atrial or ventricular depolarizations (e.g., P- or R-waves) and indicate the existence of the atrial depolarization (e.g., P-waves) or ventricular depolarizations (e.g., R-waves) to the control circuit.

The device 10 and the separate medical device 50 may cooperate to provide cardiac therapy to the patient's heart 8. For example, the device 10 and the separate medical device 50 may be used to detect tachycardia, monitor tachycardia, and/or provide tachycardia-related therapy. For example, the device 10 may communicate with the separate medical device 50 wirelessly to trigger shock therapy using the separate medical device 50. As used herein, “wirelessly” refers to an operative coupling or connection without using a metal conductor between the device 10 and the separate medical device 50. In one example, wireless communication may use a distinctive, signaling, or triggering electrical pulse provided by the device 10 that conducts through the patient's tissue and is detectable by the separate medical device 50. In another example, wireless communication may use a communication interface (e.g., an antenna) of the device 10 to provide electromagnetic radiation that propagates through patient's tissue and is detectable, for example, using a communication interface (e.g., an antenna) of the separate medical device 50.

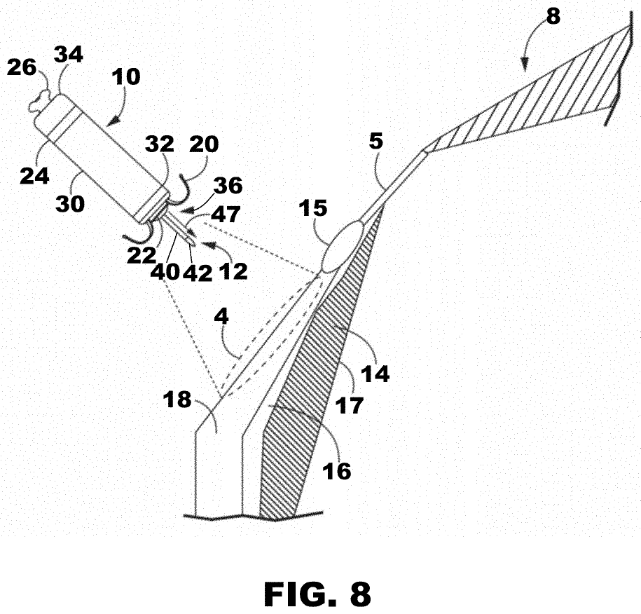

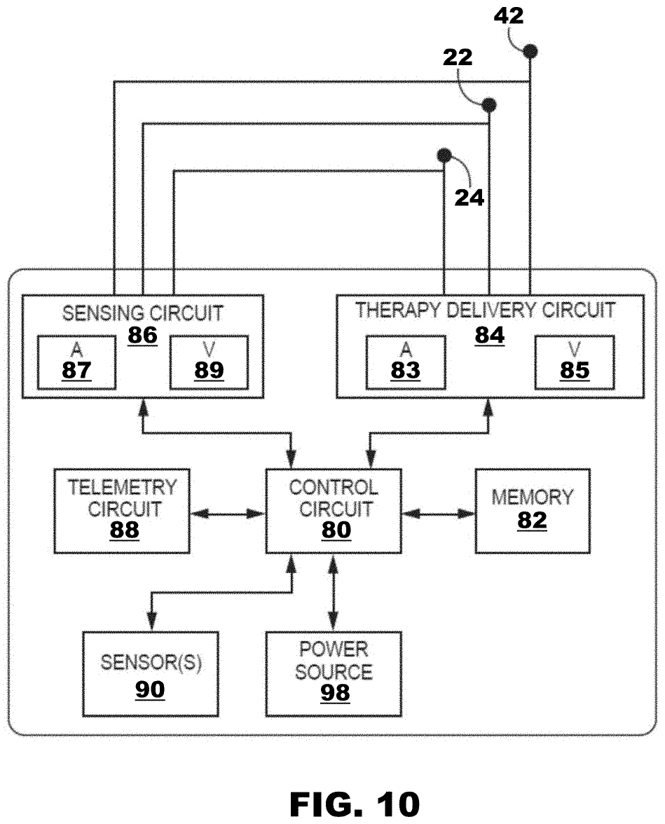

FIG. 8 is an enlarged conceptual diagram of the intracardiac medical device 10 of FIG. 7 and anatomical structures of the patient's heart 8. In particular, the device 10 is configured to sense cardiac signals and/or deliver pacing therapy. The intracardiac device 10 may include a housing 30. The housing 30 may define a hermetically sealed internal cavity in which internal components of the device 10 reside, such as a sensing circuit, therapy delivery circuit, control circuit, memory, telemetry circuit, other optional sensors, and a power source as generally described in conjunction with FIG. 10. The housing 30 may include (e.g., be formed of or from) an electrically conductive material such as, e.g., titanium or titanium alloy, stainless steel, MP35N (a non-magnetic nickel-cobalt-chromium-molybdenum alloy), platinum alloy, or other bio-compatible metal or metal alloy. In other examples, the housing 30 may include (e.g., be formed of or from) a non-conductive material including ceramic, glass, sapphire, silicone, polyurethane, epoxy, acetyl co-polymer plastics, polyether ether ketone (PEEK), a liquid crystal polymer, or other biocompatible polymer.

In at least one embodiment, the housing 30 may be described as extending between a distal end region 32 and a proximal end region 34 and as defining a generally cylindrical shape, e.g., to facilitate catheter delivery. In other embodiments, the housing 30 may be prismatic or any other shape to perform the functionality and utility described herein. The housing 30 may include a delivery tool interface member 26, e.g., defined, or positioned, at the proximal end region 34, for engaging with a delivery tool during implantation of the device 10.

All or a portion of the housing 30 may function as a sensing and/or pacing electrode during cardiac therapy. In the example shown, the housing 30 includes a proximal housing-based electrode 24 that circumscribes a proximal portion (e.g., closer to the proximal end region 34 than the distal end region 32) of the housing 30. When the housing 30 is (e.g., defines, formed from, etc.) an electrically-conductive material, such as a titanium alloy or other examples listed above, portions of the housing 30 may be electrically insulated by a non-conductive material, such as a coating of parylene, polyurethane, silicone, epoxy, or other biocompatible polymer, leaving one or more discrete areas of conductive material exposed to form, or define, the proximal housing-based electrode 24. When the housing 30 is (e.g., defines, formed from, etc.) a non-conductive material, such as a ceramic, glass or polymer material, an electrically conductive coating or layer, such as a titanium, platinum, stainless steel, or alloys thereof, may be applied to one or more discrete areas of the housing 30 to form, or define, the proximal housing-based electrode 24. In other examples, the proximal housing-based electrode 24 may be a component, such as a ring electrode, that is mounted or assembled onto the housing 30. The proximal housing-based electrode 24 may be electrically coupled to internal circuitry of the device 10, e.g., via the electrically conductive housing 30 or an electrical conductor when the housing 30 is a non-conductive material.

In the example shown, the proximal housing-based electrode 24 is located nearer to the housing proximal end region 34 than the housing distal end region 32, and therefore, may be referred to as a proximal housing-based electrode 24. In other examples, however, the proximal housing-based electrode 24 may be located at other positions along the housing 30, e.g., more distal relative to the position shown.

At the distal end region 32, the device 10 may include a distal fixation and electrode assembly 36, which may include one or more fixation members 20 and one or more dart electrode assemblies 12 of equal or unequal length. In one such example as shown, a single dart electrode assembly 12 includes a shaft 40 extending distally away from the housing distal end region 32 and one or more electrode elements, such as a tip electrode 42 at or near the free, distal end region of the shaft 40. The tip electrode 42 may have a conical or hemi-spherical distal tip with a relatively narrow tip-diameter (e.g., less than about 1 millimeter (mm)) for penetrating into and through tissue layers without using a sharpened tip or needle-like tip having sharpened or beveled edges.

The dart electrode assembly 12 may be configured to pierce through one or more tissue layers to position the tip electrode 42 within a desired tissue layer such as, e.g., the ventricular myocardium. As such, the height 47, or length, of the shaft 40 may correspond to the expected pacing site depth, and the shaft 40 may have a relatively high compressive strength along its longitudinal axis to resist bending in a lateral or radial direction when pressed against and into the implant region 4. If a second dart electrode assembly 12 is employed, its length may be unequal to the expected pacing site depth and may be configured to act as an indifferent electrode for delivering of pacing energy to and/or sensing signals from the tissue. In one embodiment, a longitudinal axial force may be applied against the tip electrode 42, e.g., by applying longitudinal pushing force to the proximal end 34 of the housing 30, to advance the dart electrode assembly 12 into the tissue within the target implant region.

The shaft 40 may be described as longitudinally non-compressive and/or elastically deformable in lateral or radial directions when subjected to lateral or radial forces to allow temporary flexing, e.g., with tissue motion, but may return to its normally straight position when lateral forces diminish. Thus, the dart electrode assembly 12 including the shaft 40 may be described as being resilient. When the shaft 40 is not exposed to any external force, or to only a force along its longitudinal central axis, the shaft 40 may retain a straight, linear position as shown.

In other words, the shaft 40 of the dart electrode assembly 12 may be a normally straight member and may be rigid. In other embodiments, the shaft 40 may be described as being relatively stiff but still possessing limited flexibility in lateral directions. Further, the shaft 40 may be non-rigid to allow some lateral flexing with heart motion. However, in a relaxed state, when not subjected to any external forces, the shaft 40 may maintain a straight position as shown to hold the tip electrode 42 spaced apart from the housing distal end region 32 at least by a height, or length, 47 of the shaft 40.

The one or more fixation members 20 may be described as one or more “tines” having a normally curved position. The tines may be held in a distally extended position within a delivery tool. The distal tips of tines may penetrate the heart tissue to a limited depth before elastically, or resiliently, curving back proximally into the normally curved position (shown) upon release from the delivery tool. Further, the fixation members 20 may include one or more aspects described in, for example, U.S. Pat. No. 9,675,579 (Grubac et al.), issued 13 Jun. 2017, and U.S. Pat. No. 9,119,959 (Rys et al.), issued 1 Sep. 2015, each of which is incorporated herein by reference in its entirety.

In some examples, the distal fixation and electrode assembly 36 includes a distal housing-based electrode 22. In the case of using the device 10 as a pacemaker for multiple chamber pacing (e.g., dual or triple chamber pacing) and sensing, the tip electrode 42 may be used as a cathode electrode paired with the proximal housing-based electrode 24 serving as a return anode electrode. Alternatively, the distal housing-based electrode 22 may serve as a return anode electrode paired with tip electrode 42 for sensing ventricular signals and delivering ventricular pacing pulses. In other examples, the distal housing-based electrode 22 may be a cathode electrode for sensing atrial signals and delivering pacing pulses to the atrial myocardium in the target implant region 4. When the distal housing-based electrode 22 serves as an atrial cathode electrode, the proximal housing-based electrode 24 may serve as the return anode paired with the tip electrode 42 for ventricular pacing and sensing and as the return anode paired with the distal housing-based electrode 22 for atrial pacing and sensing.

As shown in this illustration, the target implant region 4 in some pacing applications is along the atrial endocardium 18, generally inferior to the AV node 15 and the His bundle 5. The dart electrode assembly 12 may at least partially define the height 47, or length, of the shaft 40 for penetrating through the atrial endocardium 18 in the target implant region 4, through the central fibrous body 16, and into the ventricular myocardium 14 without perforating through the ventricular endocardial surface 17. When the height 47, or length, of the dart electrode assembly 12 is fully advanced into the target implant region 4, the tip electrode 42 may rest within the ventricular myocardium 14, and the distal housing-based electrode 22 may be positioned in intimate contact with or close proximity to the atrial endocardium 18. The dart electrode assembly 12 may have a total combined height 47, or length, of tip electrode 42 and shaft 40 from about 3 mm to about 8 mm in various examples. The diameter of the shaft 40 may be less than about 2 mm, and may be about 1 mm or less, or even about 0.6 mm or less.

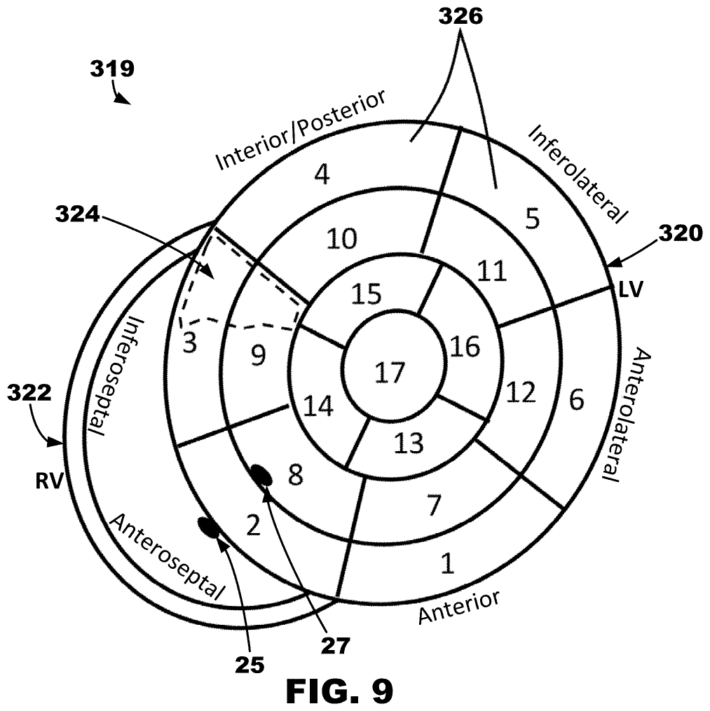

FIG. 9 is a two-dimensional (2D) ventricular map 300 of a patient's heart (e.g., a top-down view) showing the left ventricle 320 in a standard 17 segment view and the right ventricle 322. The map 300 defines, or includes, a plurality of areas 326 corresponding to different regions of a human heart. As illustrated, the areas 326 are numerically labeled 1-17 (which, e.g., correspond to a standard 17 segment model of a human heart, correspond to 17 segments of the left ventricle of a human heart, etc.). Areas 326 of the map 300 may include basal anterior area 1, basal anteroseptal area 2, basal inferoseptal area 3, basal inferior area 4, basal inferolateral area 5, basal anterolateral area 6, mid-anterior area 7, mid-anteroseptal area 8, mid-inferoseptal area 9, mid-inferior area 10, mid-inferolateral area 11, mid-anterolateral area 12, apical anterior area 13, apical septal area 14, apical inferior area 15, apical lateral area 16, and apex area 17. The inferoseptal and anteroseptal areas of the right ventricle 322 are also illustrated, as well as the right bunch branch (RBB) 25 and left bundle branch (LBB) 27.

In some embodiments, any of the tissue-piercing electrodes of the present disclosure may be implanted in the basal and/or septal region of the left ventricular myocardium of the patient's heart. In particular, the tissue-piercing electrode may be implanted from the triangle of Koch region of the right atrium through the right atrial endocardium and central fibrous body. Once implanted, the tissue-piercing electrode may be positioned in the target implant region 4 (FIGS. 7-8), such as the basal and/or septal region of the left ventricular myocardium. With reference to map 300, the basal region includes one or more of the basal anterior area 1, basal anteroseptal area 2, basal inferoseptal area 3, basal inferior area 4, mid-anterior area 7, mid-anteroseptal area 8, mid-inferoseptal area 9, and mid-inferior area 10. With reference to map 300, the septal region includes one or more of the basal anteroseptal area 2, basal anteroseptal area 3, mid-anteroseptal area 8, mid-inferoseptal area 9, and apical septal area 14.

In some embodiments, the tissue-piercing electrode may be positioned in the basal septal region of the left ventricular myocardium when implanted and may be configured to deliver pacing therapy to the left ventricular myocardium as well as one or more areas of the cardiac conduction system including, but not limited to areas of the left bundle branches and the right bundle branches. The basal septal region may include one or more of the basal anteroseptal area 2, basal inferoseptal area 3, mid-anteroseptal area 8, and mid-inferoseptal area 9.

In some embodiments, the tissue-piercing electrode may be positioned in the high inferior/posterior basal septal region of the left ventricular myocardium when implanted. The high inferior/posterior basal septal region of the left ventricular myocardium may include a portion of one or more of the basal inferoseptal area 3 and mid-inferoseptal area 9 (e.g., the basal inferoseptal area only, the mid-inferoseptal area only, or both the basal inferoseptal area and the mid-inferoseptal area). For example, the high inferior/posterior basal septal region may include region 324 illustrated generally as a dashed-line boundary. As shown, the dashed line boundary represents an approximation of where the high inferior/posterior basal septal region is located, which may take a somewhat different shape or size depending on the particular application.

A block diagram of circuitry is depicted in FIG. 10 that may be enclosed within the housings 30 of the device 10 to provide the functions of sensing cardiac signals, determining capture, and/or delivering pacing therapy according to one example or within the housings of any other medical devices described herein. The separate medical device 50 as shown in FIG. 7 may include some or all the same components, which may be configured in a similar manner. The electronic circuitry enclosed within the housing 30 may include software, firmware, and hardware that cooperatively monitor atrial and ventricular electrical cardiac signals, determine whether cardiac system capture has occurred, determine when a cardiac therapy is necessary, and/or deliver electrical pulses to the patient's heart according to programmed therapy mode and pulse control parameters. The electronic circuitry may include a control circuit 80 (e.g., including processing circuitry), a memory 82, a therapy delivery circuit 84, a sensing circuit 86, and/or a telemetry circuit 88. In some examples, the device 10 includes one or more sensors 90 for producing signals that are correlated to one or more physiological functions, states, or conditions of the patient. For example, the sensor(s) 90 may include a patient activity sensor, for use in determining a need for pacing therapy and/or controlling a pacing rate. In other words, the device 10 may include other sensors 90 for sensing signals from the patient for use in determining whether to deliver and/or controlling electrical stimulation therapies delivered by the therapy delivery circuit 84.

The power source 98 may provide power to the circuitry of the device 10 including each of the components 80, 82, 84, 86, 88, 90 as needed. The power source 98 may include one or more energy storage devices, such as one or more rechargeable or non-rechargeable batteries. The connections (not shown) between the power source 98 and each of the components 80, 82, 84, 86, 88, 90 may be understood from the general block diagram illustrated to one of ordinary skill in the art. For example, the power source 98 may be coupled to one or more charging circuits included in the therapy delivery circuit 84 for providing the power used to charge holding capacitors included in the therapy delivery circuit 84 that are discharged at appropriate times under the control of the control circuit 80 for delivering pacing pulses, e.g., according to a dual chamber pacing mode such as DDI(R). The power source 98 may also be coupled to components of the sensing circuit 86, such as sense amplifiers, analog-to-digital converters, switching circuitry, etc., sensors 90, the telemetry circuit 88, and the memory 82 to provide power to the various circuits.

The functional blocks shown in FIG. 10 represent functionality included in the device 10 and may include any discrete and/or integrated electronic circuit components that implement analog, and/or digital circuits capable of producing the functions attributed to the medical device 10 described herein. The various components may include processing circuitry, such as an application specific integrated circuit (ASIC), an electronic circuit, a processor (shared, dedicated, or group), and memory that execute one or more software or firmware programs, a combinational logic circuit, state machine, or other suitable components or combinations of components that provide the described functionality. The particular form of software, hardware, and/or firmware employed to implement the functionality disclosed herein will be determined primarily by the particular system architecture employed in the medical device and by the particular detection and therapy delivery methodologies employed by the medical device.

The memory 82 may include any volatile, non-volatile, magnetic, or electrical non-transitory computer readable storage media, such as random-access memory (RAM), read-only memory (ROM), non-volatile RAM (NVRAM), electrically erasable programmable ROM (EEPROM), flash memory, or any other memory device. Furthermore, the memory 82 may include a non-transitory computer readable media storing instructions that, when executed by one or more processing circuits, cause the control circuit 80 and/or other processing circuitry to determine posterior left bundle branch engagement and/or perform a single, dual, or triple chamber calibrated pacing therapy (e.g., single or multiple chamber pacing), or other cardiac therapy functions (e.g., sensing or delivering therapy), attributed to the device 10. The non-transitory computer-readable media storing the instructions may include any of the media listed above.

The control circuit 80 may communicate, e.g., via a data bus, with the therapy delivery circuit 84 and the sensing circuit 86 for sensing cardiac electrical signals and controlling delivery of cardiac electrical stimulation therapies in response to sensed cardiac events, e.g., P-waves and R-waves, or the absence thereof. The tip electrode 42, the distal housing-based electrode 22, and the proximal housing-based electrode 24 may be electrically coupled to the therapy delivery circuit 84 for delivering electrical stimulation pulses to the patient's heart and to the sensing circuit 86 and for sensing cardiac electrical signals.

The sensing circuit 86 may include an atrial (A) sensing channel 87 and a ventricular (V) sensing channel 89. The distal housing-based electrode 22 and the proximal housing-based electrode 24 may be coupled to the atrial sensing channel 87 for sensing atrial signals, e.g., P-waves attendant to the depolarization of the atrial myocardium. In examples that include two or more selectable distal housing-based electrodes, the sensing circuit 86 may include switching circuitry for selectively coupling one or more of the available distal housing-based electrodes to cardiac event detection circuitry included in the atrial sensing channel 87. Switching circuitry may include a switch array, switch matrix, multiplexer, or any other type of switching device suitable to selectively couple components of the sensing circuit 86 to selected electrodes. The tip electrode 42 and the proximal housing-based electrode 24 may be coupled to the ventricular sensing channel 89 for sensing ventricular signals, e.g., R-waves attendant to the depolarization of the ventricular myocardium.

Each of the atrial sensing channel 87 and the ventricular sensing channel 89 may include cardiac event detection circuitry for detecting P-waves and R-waves, respectively, from the cardiac electrical signals received by the respective sensing channels. The cardiac event detection circuitry included in each of the channels 87 and 89 may be configured to amplify, filter, digitize, and rectify the cardiac electrical signal received from the selected electrodes to improve the signal quality for detecting cardiac electrical events. The cardiac event detection circuitry within each channel 87 and 89 may include one or more sense amplifiers, filters, rectifiers, threshold detectors, comparators, analog-to-digital converters (ADCs), timers, or other analog or digital components. A cardiac event sensing threshold, e.g., a P-wave sensing threshold and an R-wave sensing threshold, may be automatically adjusted by each respective sensing channel 87 and 89 under the control of the control circuit 80, e.g., based on timing intervals and sensing threshold values determined by the control circuit 80, stored in the memory 82, and/or controlled by hardware, firmware, and/or software of the control circuit 80 and/or the sensing circuit 86.

Upon detecting a cardiac electrical event based on a sensing threshold crossing, the sensing circuit 86 may produce a sensed event signal that is passed to the control circuit 80. For example, the atrial sensing channel 87 may produce a P-wave sensed event signal in response to a P-wave sensing threshold crossing. The ventricular sensing channel 89 may produce an R-wave sensed event signal in response to an R-wave sensing threshold crossing. The sensed event signals may be used by the control circuit 80 for setting pacing escape interval timers that control the basic time intervals used for scheduling cardiac pacing pulses. A sensed event signal may trigger or inhibit a pacing pulse depending on the particular programmed pacing mode. For example, a P-wave sensed event signal received from the atrial sensing channel 87 may cause the control circuit 80 to inhibit a scheduled atrial pacing pulse and schedule a ventricular pacing pulse at a programmed atrioventricular (A-V) pacing interval. If an R-wave is sensed before the A-V pacing interval expires, the ventricular pacing pulse may be inhibited. If the A-V pacing interval expires before the control circuit 80 receives an R-wave sensed event signal from the ventricular sensing channel 89, the control circuit 80 may use the therapy delivery circuit 84 to deliver the scheduled ventricular pacing pulse synchronized to the sensed P-wave.

In some examples, the device 10 may be configured to deliver a variety of pacing therapies including bradycardia pacing, cardiac resynchronization therapy, post-shock pacing, and/or tachycardia-related therapy, such as ATP, among others. For example, the device 10 may be configured to detect non-sinus tachycardia and deliver ATP. The control circuit 80 may determine cardiac event time intervals, e.g., P-P intervals between consecutive P-wave sensed event signals received from the atrial sensing channel 87, R-R intervals between consecutive R-wave sensed event signals received from the ventricular sensing channel 89, and P-R and/or R-P intervals received between P-wave sensed event signals and R-wave sensed event signals. These intervals may be compared to tachycardia detection intervals for detecting non-sinus tachycardia. Tachycardia may be detected in a given heart chamber based on a threshold number of tachycardia detection intervals being detected.

The therapy delivery circuit 84 may include atrial pacing circuit 83 and ventricular pacing circuit 85. Each pacing circuit 83, 85 may include charging circuitry, one or more charge storage devices such as one or more low voltage holding capacitors, an output capacitor, and/or switching circuitry that controls when the holding capacitor(s) are charged and discharged across the output capacitor to deliver a pacing pulse to the pacing electrode vector coupled to respective pacing circuits 83, 85. The tip electrode 42 and the proximal housing-based electrode 24 may be coupled to the ventricular pacing circuit 85 as a bipolar cathode and anode pair for delivering ventricular pacing pulses, e.g., upon expiration of an A-V or V-V pacing interval set by the control circuit 80 for providing atrial-synchronized ventricular pacing and a basic lower ventricular pacing rate.

The atrial pacing circuit 83 may be coupled to the distal housing-based electrode 22 and the proximal housing-based electrode 24 to deliver atrial pacing pulses. The control circuit 80 may set one or more atrial pacing intervals according to a programmed lower pacing rate or a temporary lower rate set according to a rate-responsive sensor indicated pacing rate. Atrial pacing circuit may be controlled to deliver an atrial pacing pulse if the atrial pacing interval expires before a P-wave sensed event signal is received from the atrial sensing channel 87. The control circuit 80 starts an A-V pacing interval in response to a delivered atrial pacing pulse to provide synchronized multiple chamber pacing (e.g., dual or triple chamber pacing).

Charging of a holding capacitor of the atrial or ventricular pacing circuit 83, 85 to a programmed pacing voltage amplitude and discharging of the capacitor for a programmed pacing pulse width may be performed by the therapy delivery circuit 84 according to control signals received from the control circuit 80. For example, a pace timing circuit included in the control circuit 80 may include programmable digital counters set by a microprocessor of the control circuit 80 for controlling the basic pacing time intervals associated with various single chamber or multiple chamber pacing (e.g., dual or triple chamber pacing) modes or anti-tachycardia pacing sequences. The microprocessor of the control circuit 80 may also set the amplitude, pulse width, polarity, or other characteristics of the cardiac pacing pulses, which may be based on programmed values stored in the memory 82.

Control parameters utilized by the control circuit 80 for sensing cardiac events and controlling pacing therapy delivery may be programmed into the memory 82 via the telemetry circuit 88, which may also be described as a communication interface. The telemetry circuit 88 includes a transceiver and antenna for communicating with an external device, such as a programmer or home monitor, using radio frequency communication or other communication protocols. The control circuit 80 may use the telemetry circuit 88 to receive downlink telemetry from and send uplink telemetry to the external device. In some cases, the telemetry circuit 88 may be used to transmit and receive communication signals to/from another medical device implanted in the patient.

The techniques described in this disclosure, including those attributed to the IMD 10, device 50, the computing apparatus 140, and the computing device 160 and/or various constituent components, may be implemented, at least in part, in hardware, software, firmware, or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, stimulators, image processing devices, or other devices. The term “module,” “processor,” or “processing circuitry” may generally refer to any of the foregoing logic circuitry, alone or in combination with other logic circuitry, or any other equivalent circuitry.

Such hardware, software, and/or firmware may be implemented within the same device or within separate devices to support the various operations and functions described in this disclosure. In addition, any of the described units, modules, or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components.

When implemented in software, the functionality ascribed to the systems, devices and techniques described in this disclosure may be embodied as instructions on a computer-readable medium such as RAM, ROM, NVRAM, EEPROM, FLASH memory, magnetic data storage media, optical data storage media, or the like. The instructions may be executed by processing circuitry and/or one or more processors to support one or more aspects of the functionality described in this disclosure.

All references and publications cited herein are expressly incorporated herein by reference in their entirety for all purposes, except to the extent any aspect incorporated directly contradicts this disclosure.

All scientific and technical terms used herein have meanings commonly used in the art unless otherwise specified. The definitions provided herein are to facilitate understanding of certain terms used frequently herein and are not meant to limit the scope of the present disclosure.

Unless otherwise indicated, all numbers expressing feature sizes, amounts, and physical properties used in the specification and claims may be understood as being modified either by the term “exactly” or “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein or, for example, within typical ranges of experimental error.

The recitation of numerical ranges by endpoints includes all numbers subsumed within that range (e.g., 1 to 5 includes 1, 1.5, 2, 2.75, 3, 3.80, 4, and 5) and any range within that range. Herein, the terms “up to” or “no greater than” a number (e.g., up to 50) includes the number (e.g., 50), and the term “no less than” a number (e.g., no less than 5) includes the number (e.g., 5).

The terms “coupled” or “connected” refer to elements being attached to each other either directly (in direct contact with each other) or indirectly (having one or more elements between and attaching the two elements). Either term may be modified by “operatively” and “operably,” which may be used interchangeably, to describe that the coupling or connection is configured to allow the components to interact to carry out at least some functionality (for example, a first medical device may be operatively coupled to another medical device to transmit information in the form of data or to receive data therefrom).

Terms related to orientation, such as “top,” “bottom,” “side,” and “end,” are used to describe relative positions of components and are not meant to limit the orientation of the embodiments contemplated. For example, an embodiment described as having a “top” and “bottom” also encompasses embodiments thereof rotated in various directions unless the content clearly dictates otherwise.

Reference to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout are not necessarily referring to the same embodiment of the disclosure. Furthermore, the particular features, configurations, compositions, or characteristics may be combined in any suitable manner in one or more embodiments.

As used in this specification and the appended claims, the singular forms “a,” “an,” and “the” encompass embodiments having plural referents, unless the content clearly dictates otherwise. As used in this specification and the appended claims, the term “or” is generally employed in its sense including “and/or” unless the content clearly dictates otherwise.

As used herein, “have,” “having,” “include,” “including,” “comprise,” “comprising” or the like are used in their open-ended sense, and generally mean “including, but not limited to.” It will be understood that “consisting essentially of” “consisting of” and the like are subsumed in “comprising,” and the like.

The term “and/or” means one or all the listed elements or a combination of at least two of the listed elements. The phrases “at least one of,” “comprises at least one of” and “one or more of” followed by a list refers to any one of the items in the list and any combination of two or more items in the list.

### ILLUSTRATIVE EMBODIMENTS

Embodiment 1: A system comprising:
a computing apparatus comprising processing circuitry and configured to:
obtain external electrical activity measured from tissue of a patient during intrinsic activation of the patient's heart,
generate electrical heterogeneity information (EHI) based on the obtained electrical activity, wherein the EHI comprises one or more metrics of dispersion of cardiac electrical activation times, and
determine that cardiac conduction system pacing therapy would benefit the patient based on at least the one or more metrics of dispersion of cardiac electrical activation times.

### Embodiment 2: A method comprising:

obtaining external electrical activity measured from tissue of a patient during intrinsic activation of the patient's heart;
generating electrical heterogeneity information (EHI) based on the obtained electrical activity, wherein the EHI comprises one or more metrics of dispersion of cardiac electrical activation times; and
determining that cardiac conduction method pacing therapy would benefit the patient based on at least the one or more metrics of dispersion of cardiac electrical activation times.

Embodiment 3: The system as in embodiment 1 or method as in embodiment 2, wherein the one or more metrics of dispersion of cardiac electrical activation times comprise a standard deviation of activation times (SDAT) of the obtained external electrical activity.

Embodiment 4: The system or method as in embodiment 3, wherein determining that cardiac conduction system pacing therapy would benefit the patient based on at least the one or more metrics of dispersion of cardiac electrical activation times comprises determining that cardiac conduction system pacing therapy would benefit the patient if the SDAT is greater than or equal to a SDAT threshold.

Embodiment 5: The system or method as in embodiment 4, wherein the SDAT threshold is greater than or equal to 40 milliseconds.

Embodiment 6: The system or method as in any one of embodiments 1-5, wherein the one or more metrics of dispersion of cardiac electrical activation times comprise a left-sided metric of dispersion of the obtained external electrical activity from the left side of the patient's torso.

Embodiment 7: The system or method as in embodiment 6, wherein the left-sided metric of dispersion comprises a left-sided standard deviation of activation times (LVED) of the obtained external electrical activity from the left side of the patient's torso.

Embodiment 8: The system or method as in any one of embodiments 6-7, wherein determining that cardiac conduction system pacing therapy would benefit the patient based on at least the one or more metrics of dispersion of cardiac electrical activation times comprises determining that cardiac conduction system pacing therapy would benefit the patient if the left-sided metric of dispersion is greater than or equal to a left-sided dispersion threshold.

Embodiment 9: The system or method as in embodiment 8, wherein the left-sided dispersion threshold is greater than or equal to 30 milliseconds.

Embodiment 10: The system or method as in any one of embodiments 1-9, wherein the computing apparatus is further configured to execute or the method further comprises determining that myocardial pacing therapy would benefit the patient based on at least the one or more metrics of dispersion of cardiac electrical activation times.

Embodiment 11: The system or method as in any one of embodiments 1-10, wherein the system further comprises a display apparatus comprising a graphical user interface, wherein the computing apparatus is operably coupled to the display apparatus, wherein computing apparatus is further configured to execute or the method further comprises displaying, on a graphical user interface, one or more of an indication of the determination that cardiac conduction system pacing therapy would benefit the patient and the one or more metrics of dispersion of cardiac electrical activation times.

Embodiment 12: The system or method as in any one of embodiments 1-10, wherein the system further comprises or the method further comprises providing electrode apparatus comprising a plurality of external electrodes to be located proximate the skin of the torso of the patient to measure the external electrical activity.

Embodiment 13: A system comprising:
a computing apparatus comprising processing circuitry and configured to:
generate one or more metrics of dispersion of cardiac electrical activation times based on a plurality of surrogate cardiac electrical activation times representative of depolarization of a plurality of regions of a patient's heart during intrinsic activation, and
determine that cardiac conduction system pacing therapy would benefit the patient based on at least the one or more metrics of dispersion of cardiac electrical activation times.

This disclosure has been provided with reference to illustrative embodiments and is not meant to be construed in a limiting sense. As described previously, one skilled in the art will recognize that other various illustrative applications may use the techniques as described herein to take advantage of the beneficial characteristics of the apparatus and methods described herein. Various modifications of the illustrative embodiments, as well as additional embodiments of the disclosure, will be apparent upon reference to this description.

What is claimed is:

1. A system comprising:
a computing apparatus comprising processing circuitry and configured to:
obtain external electrical activity measured from tissue of a patient during intrinsic activation of the patient's heart,
generate electrical heterogeneity information (EHI) based on the obtained electrical activity, wherein the EHI comprises one or more metrics of dispersion of cardiac electrical activation times, and
determine that cardiac conduction system pacing therapy would benefit the patient based on at least the one or more metrics of dispersion of cardiac electrical activation times.

2. The system of claim 1, wherein the one or more metrics of dispersion of cardiac electrical activation times comprise a standard deviation of activation times (SDAT) of the obtained external electrical activity.

3. The system of claim 2, wherein determining that cardiac conduction system pacing therapy would benefit the patient based on at least the one or more metrics of dispersion of cardiac electrical activation times comprises determining that cardiac conduction system pacing therapy would benefit the patient if the SDAT is greater than or equal to a SDAT threshold.

4. The system of claim 3, wherein the SDAT threshold is greater than or equal to 40 milliseconds.

5. The system of claim 1, wherein the one or more metrics of dispersion of cardiac electrical activation times comprise a left-sided metric of dispersion of the obtained external electrical activity from the left side of the patient's torso.

6. The system of claim 5, wherein the left-sided metric of dispersion comprises a left-sided standard deviation of activation times (LVED) of the obtained external electrical activity from the left side of the patient's torso.

7. The system of claim 5, wherein determining that cardiac conduction system pacing therapy would benefit the patient based on at least the one or more metrics of dispersion of cardiac electrical activation times comprises determining that cardiac conduction system pacing therapy would benefit the patient if the left-sided metric of dispersion is greater than or equal to a left-sided dispersion threshold.

8. The system of claim 7, wherein the left-sided dispersion threshold is greater than or equal to 30 milliseconds.

9. The system of claim 1, wherein the computing apparatus is further configured to determine that conventional myocardial pacing therapy would benefit the patient based on at least the one or more metrics of dispersion of cardiac electrical activation times.

10. The system of claim 1, further comprising a display apparatus comprising a graphical user interface, wherein the computing apparatus is operably coupled to the display apparatus and further configured to display, on the graphical user interface, one or more of an indication of the determination that cardiac conduction system pacing therapy would benefit the patient and the one or more metrics of dispersion of cardiac electrical activation times.

11. The system of claim 1, further comprising electrode apparatus comprising a plurality of external electrodes to be located proximate the skin of the torso of the patient to measure the external electrical activity.

12. A method comprising:
obtaining external electrical activity measured from tissue of a patient during intrinsic activation of the patient's heart;
generating electrical heterogeneity information (EHI) based on the obtained electrical activity, wherein the EHI comprises one or more metrics of dispersion of cardiac electrical activation times; and
determining that cardiac conduction method pacing therapy would benefit the patient based on at least the one or more metrics of dispersion of cardiac electrical activation times.

13. The method of claim 12, wherein the one or more metrics of dispersion of cardiac electrical activation times comprise a standard deviation of activation times (SDAT) of the obtained external electrical activity.

14. The method of claim 13, wherein determining that cardiac conduction method pacing therapy would benefit the patient based on at least the one or more metrics of dispersion of cardiac electrical activation times comprises determining that cardiac conduction method pacing therapy would benefit the patient if the SDAT is greater than or equal to a SDAT threshold.

15. The method of claim 14, wherein the SDAT threshold is greater than or equal to 40 milliseconds.

16. The method of claim 12, wherein the one or more metrics of dispersion of cardiac electrical activation times comprise a left-sided metric of dispersion of the obtained external electrical activity from the left side of the patient's torso.

17. The method of claim 16, wherein the left-sided metric of dispersion comprises a left-sided standard deviation of activation times (LVED) of the obtained external electrical activity from the left side of the patient's torso.

18. The method of claim 16, wherein determining that cardiac conduction method pacing therapy would benefit the patient based on at least the one or more metrics of dispersion of cardiac electrical activation times comprises determining that cardiac conduction method pacing therapy would benefit the patient if the left-sided metric of dispersion is greater than or equal to a left-sided dispersion threshold.

19. The method of claim 18, wherein the left-sided dispersion threshold is greater than or equal to 30 milliseconds.

20. The method of claim 12, the method further comprising determining that conventional myocardial pacing therapy would benefit the patient based on at least the one or more metrics of dispersion of cardiac electrical activation times.

21. The method of claim 12, further comprising displaying, on a graphical user interface, one or more of an indication of the determination that cardiac conduction method pacing therapy would benefit the patient and the one or more metrics of dispersion of cardiac electrical activation times.

22. The method of claim 12, wherein obtaining external electrical activity measured from tissue of a patient during intrinsic activation of the patient's heart comprises measuring external electrical activity using electrode apparatus comprising a plurality of external electrodes to be located proximate the skin of the torso of the patient.

23. A system comprising:
a computing apparatus comprising processing circuitry and configured to:
generate one or more metrics of dispersion of cardiac electrical activation times based on a plurality of surrogate cardiac electrical activation times representative of depolarization of a plurality of regions of a patient's heart during intrinsic activation, and
determine that cardiac conduction system pacing therapy would benefit the patient based on at least the one or more metrics of dispersion of cardiac electrical activation times.
